SEQUENCE LISTING (31 sequences)

SEQ ID NO: 1 - protein, 5 aa, synthetic construct
  REGION 1..5: note = CDR1 = hypervariable region 1 of heavy chain of AIN457
NYWMN

SEQ ID NO: 2 - protein, 17 aa, synthetic construct
  REGION 1..17: note = CDR2 = hypervariable region 2 of heavy chain of AIN457
AINQDGSEKYYVGSVKG

SEQ ID NO: 3 - protein, 18 aa, synthetic construct
  REGION 1..18: note = CDR3 = hypervariable region 3 of heavy chain of AIN457
DYYDILTDYYIHYWYFDL

SEQ ID NO: 4 - protein, 12 aa, synthetic construct
  REGION 1..12: note = CDR1' = hypervariable region 1 of light chain of AIN457
RASQSVSSSYLA

SEQ ID NO: 5 - protein, 7 aa, synthetic construct
  REGION 1..7: note = CDR2' = hypervariable region 2 of light chain AIN457
GASSRAT

SEQ ID NO: 6 - protein, 9 aa, synthetic construct
  REGION 1..9: note = CDR3' = hypervariable region 3 of light chain AIN457
QQYGSSPCT

SEQ ID NO: 7 - DNA, 381 bp, Homo sapiens
  source 1..381: mol_type = unassigned DNA
  CDS 1..381
gaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctca

SEQ ID NO: 8 - protein, 127 aa, Homo sapiens [sequence omitted]

SEQ ID NO: 9 - DNA, 327 bp, Homo sapiens
  source 1..327: mol_type = unassigned DNA
  CDS 1..327
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacga

SEQ ID NO: 10 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 11 - protein, 10 aa, synthetic construct
  REGION 1..10: note = CDR1-x = hypervariable domain x of heavy chain of AIN457
GFTFSNYWMN

SEQ ID NO: 12 - protein, 11 aa, synthetic construct
  REGION 1..11: note = CDR2-x = hypervariable domain of heavy chain x of AIN457
AINQDGSEKYY

SEQ ID NO: 13 - protein, 23 aa, synthetic construct
  REGION 1..23: note = CDR3-x = hypervariable domain x of heavy chain AIN457
CVRDYYDILTDYYIHYWYFDLWG

SEQ ID NO: 14 - protein, 215 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 457 aa, Homo sapiens
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 11 aa, Homo sapiens
RPSQGINWELA

SEQ ID NO: 17 - protein, 7 aa, Homo sapiens
SQGINWE

SEQ ID NO: 18 - protein, 7 aa, Homo sapiens
DASSLEQ

SEQ ID NO: 19 - skipped

SEQ ID NO: 20 - protein, 9 aa, Homo sapiens
QQFNSYPLT

SEQ ID NO: 21 - protein, 6 aa, Homo sapiens
FNSYPL

SEQ ID NO: 22 - protein, 107 aa, Homo sapiens
AIQLTQSPSSLSASVGDRVTITCRPSQGINWELAWYQQKPGKAPKLLIYDASSLEQGVPSRFSGSGSGTDFTLTISSLQPEDFATYYCQQFNSYPLTFGGGTKVEIK

SEQ ID NO: 23 - protein, 214 aa, Homo sapiens
AIQLTQSPSSLSASVGDRVTITCRPSQGINWELAWYQQKPGKAPKLLIYDASSLEQGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 5 aa, Homo sapiens
SYWMS

SEQ ID NO: 25 - protein, 7 aa, Homo sapiens
GFTFSSY

SEQ ID NO: 26 - protein, 17 aa, Homo sapiens
NIKQDGSEKYYVDSVKG

SEQ ID NO: 27 - protein, 6 aa, Homo sapiens
KQDGSE

SEQ ID NO: 28 - protein, 7 aa, Homo sapiens
DRGSLYY

SEQ ID NO: 29 - protein, 7 aa, Homo sapiens
DRGSLYY

SEQ ID NO: 30 - protein, 116 aa, Homo sapiens
EVQLVESGGDLVQPGGSLRLSCAASGFTFSSYWMSWVRQAPGKGLEWVANIKQDGSEKYYVDSVKGRFTISRDNAKNSLYLQMNSLRAEDTAVYYCARDRGSLYYWGQGTLVTVSS

SEQ ID NO: 31 - protein, 446 aa, Homo sapiens
EVQLVESGGDLVQPGGSLRLSCAASGFTFSSYWMSWVRQAPGKGLEWVANIKQDGSEKYYVDSVKGRFTISRDNAKNSLYLQMNSLRAEDTAVYYCARDRGSLYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0331834 A1
(43) Pub. Date: Oct. 19, 2023

(54) METHOD OF TREATING TENDINOPATHY USING INTERLEUKIN-17 (IL-17)

(71) Applicants: NOVARTIS AG, Basel (CH); University Court of the University of Glasgow, Glasgow (GB)

(72) Inventors: Gerard Bruin, Basel (CH); Shea Carter, Basel (CH); Frank Kolbinger, Neuenburg (DE); Iain McInnes, Glasgow (GB); Neal Millar, Glasgow (GB); Linda Mindeholm, Bettlach (FR); Matthias Schieker, Basel (CH); Eckhard Weber, Basel (CH)

(21) Appl. No.: 18/152,139

(22) Filed: Jan. 9, 2023

Related U.S. Application Data
(63) Continuation of application No. 16/760,605, filed on Apr. 30, 2020, now abandoned.
(60) Provisional application No. 62/580,715, filed on Nov. 2, 2017.

(51) Int. Cl. C07K 16/24; A61P 19/04

(52) U.S. Cl. CPC C07K 16/244; A61P 19/04; A61K 2039/505

(57) ABSTRACT

The present disclosure relates to methods for treating tendinopathy, e.g., rotator cuff tendinopathy, using IL-17 antagonists, e.g., secukinumab. Also disclosed herein are uses of IL-17 antagonists. e.g., IL-17 antibodies, such as secukinumab, for treating tendinopathy patients, as well as medicaments, dosing regimens, pharmaceutical formulations, dosage forms, and kits for use in the disclosed uses and methods.

### RELATED APPLICATIONS

The instant application claims priority to U.S. Provisional Patent application No. 62/580,715, filed Nov. 2, 2017, which is incorporated by reference herein in its entirety.

### TECHNICAL FIELD

The present disclosure relates to methods of treating tendinopathy and inducing regeneration of tendon tissue and promoting tendon repair in patients having tendinopathy using IL-17 antagonists, e.g., an IL-17 antibody or antigen-binding fragment thereof, such as secukinumab, or ixekizumab, or an IL-17 receptor antibody or antigen-binding fragment thereof, such as brodalumab.

### BACKGROUND OF THE DISCLOSURE

Overuse tendinopathy is a complex multi-faceted disease of the tendon, clinically diagnosed after gradual onset of activity-related pain, decreased function and sometimes with localized swelling of the tendon (Riley G (2005) Expert Rev Mol Med;7:1-25; Riley G (2008) Nat Clin Pract Rheumatol; 4:82-9). Historically the terms ‘tendinitis’ and ‘tendinosis’ have interchanged with the term ‘tendinopathy’, however. these definitions are now included in the spectrum of human tendon disorders (‘tendinopathy’). Tendinopathy is a common overuse injury in the athletic and working populations; it is the most common reason for consultation for a musculoskeletal complaint, corresponding to around 30% of all such consultations with a general practitioner (Forde et al (2005) J. of Occupational and Environmental Hygiene; 2:203-12; Riley (2008), supra).

The exact incidence of overuse tendon injuries is unknown, but in sports medicine, they account for 30% to 50% of all injuries (Scott and Ashe (2006) Current Sports Medicine Reports; 5:233-241). Generally, for physical workers. the prevalence of musculoskeletal symptoms increases with duration of employment (Forde et al (2005), supra).

Upper Limb Tendinopathy

A systematic review showed that the incidence of rotator cuff tendinopathy ranges from 0.3% to 5.5% per year and the annual prevalence from 0.5% to 7.4% (Littlewood et al (2013) Physiotherapy; 98:101-9). The incidence is higher in physical workers and athletes (15-20%) and in the wheelchair populations (31-73%). It is typically seen in swimmers, baseball-, tennis-, volleyball players (Kaux et al (2011) J Sports Sci Med:10:238-253).

Tennis elbow (lateral epicondylitis) is another frequent tendinopathy and is common in athletes of all ages participating in sports involving overhead or repetitive arm actions (Hume et al. (2006) Sports Medicine 36, 151-170). Its incidence in tennis players is as high as 9% to 40% (Maffulli et al (2003) Clinics in Sports Medicine 22, 675-692; Scott and Ashe (2006), supra). The condition affects approximately 1 to 3% of the general population. Another elbow tendinopathy is the golfer's elbow (medial epicondylitis), which is a typical complaint in javelin throwing, baseball and golf (Id.).

Lower Limb Tendinopathy

Achilles tendinopathy is the most prevalent lower extremity tendinopathy, with a 5.9% frequency in sedentary people and around a 50% frequency in elite endurance athletes (Scott and Ashe (2006). supra; Fredberg U and Stengaard-Pedersen K (2008) Scandinavian Journal of Medicine & Science in Sports 18, 3-15).

About one third of sports injuries treated in sports clinics concern the knees and one quarter of athletes treated for a knee injury are diagnosed with tendinopathy (Maffulli et al (2003), supra). The most common knee disorder is jumper's knee (insertional patellar tendonitis), and its incidence is reported to be in the range of 7% to 40% (Fredberg and Stengaard-Pedersen (2008). supra; Scott and Ashe (2006), supra).

Treatments

Current treatments for tendinopathy are rest, icepacks and physiotherapy (including therapeutic ultrasound, laser therapy, hyperthermia and extra corporeal shockwave therapy). Evidence to support the widespread use of these therapies in tendinopathies remains inconsistent (Alfredson and Cook (2007) Br J Sports Med: 41:211-216). Non-steroidal anti-inflammatory drugs (NSAIDS), or local corticosteroid injections can give transient pain relief, but the long term benefit is questionable (Mohamadi et al (2017) Clin. Orthop. Relat. Res.;475:232-243; Coombes et al. (2010) Lancet; 376:1751-67). Even more, it has been shown that repetitive steroid injections have the potential to accelerate tendon degeneration increasing the risk of tendon rupture (Id.). Autologous platelet rich plasma (PRP) injections are being used, however the evidence for long term efficacy is weak, thus the benefits of PRP for tendinopathy remain controversial, with several trials showing no efficacy compared with saline (Krogh TP et al (2013) Am J Sports Med 41:625-35; de Vos RJ et al (2010) JAMA: 303:144-9).

Overall. overuse tendinopathy is a disease with high medical need without adequate treatment options.

### SUMMARY OF THE DISCLOSURE

There is evidence that IL-17-expressing tendon-resident immune cells are present in human overuse tendinopathy and IL-17 mRNA and protein expression levels are increased in early human tendinopathic samples (Millar et al. (2017) Nat Rev Rheumatol;13:110-122). In human tenocytes, IL-17 regulates pro-inflammatory cytokines, key apoptotic mediators and tendon matrix changes towards a mechanically inferior type III collagen phenotype (Id.). It is postulated that IL-17, being a mediator of tendon inflammation, disrepair of tendon matrix and tenocyte apoptosis, is involved in the pathogenesis of overuse tendinopathy (Id.).

Secukinumab is a selective high-affinity fully human monoclonal antibody that neutralizes IL 17A and is approved for treating plaque psoriasis, psoriatic arthritis (PsA), and ankylosing spondylitis (AS). We have now determined that IL-17 antagonists, e.g., IL-17 antibodies. e.g., secukinumab, can be used systemically to treat tendinopathy and resolve the pain, swelling and/or loss of function associated therewith, as well as induce regeneration of tenocytes and promote tendon repair.

Accordingly, disclosed herein are methods of reducing inflammation, structural damage, and pain in the effected tendon of a tendinopathy patient, inducing regeneration of tendon tissue in a tendinopathy patient, promoting tendon repair in a tendinopathy patient, and treating a patient having tendinopathy (e.g., overuse tendinopathy), comprising administering an IL-17 antagonist (e.g., an anti-IL-17 antibody or antigen-binding fragment thereof) to a patient in need thereof.

Disclosed herein are methods, uses, pharmaceutical compositions, and kits for inducing regeneration of tendon tissue or promoting tendon repair in a patient having tendinopathy, comprising subcutaneously administering to a patient in need thereof about 150 mg-about 300 mg (e.g., a fixed dose of about 150 mg, a fixed dose of about 300 mg) of an IL-17 antibody or antigen-binding fragment thereof, wherein the IL-17 antibody or antigen-binding fragment thereof binds to an epitope of a human IL-17 homodimer having two mature human IL-17 protein chains, said epitope comprising Leu74, Tyr85, His86, Met87, Asn88, Val124. Thrl25, Pro126, Ile127, Val128, His129 on one chain and Tyr43, Tyr44, Arg46, A1a79, Asp80 on the other chain, wherein the IL-17 antibody or antigen-binding fragment thereof has a K₁ for human IL-17 of about 100-200 pM, and wherein the IL-17 antibody or antigen-binding fragment thereof has an in vivo half-life of about 4 weeks.

In some embodiments of the disclosed methods, uses, pharmaceutical compositions, and kits, the patient is administered the IL-17 antibody or antigen-binding fragment thereof only once.

In some embodiments of the disclosed methods, uses, pharmaceutical compositions, and kits, the patient is administered the IL-17 antibody or antigen-binding fragment thereof weekly.

In some embodiments of the disclosed methods, uses, pharmaceutical compositions, and kits, the patient is administered the IL-17 antibody or antigen-binding fragment thereof during week 0, 1, 2, 3, and 4.

In some embodiments of the disclosed methods, uses, pharmaceutical compositions, and kits, the patient is administered the IL-17 antibody or antigen-binding fragment thereof every 4 weeks.

In some embodiments of the disclosed methods, uses, pharmaceutical compositions, and kits, the patient is administered the IL-17 antibody or antigen-binding fragment thereof for a total treatment duration of at least two months.

In some embodiments of the disclosed methods, uses, pharmaceutical compositions, and kits, the patient is administered the IL-17 antibody or antigen-binding fragment thereof weekly during week 0, 1, 2, 3, and 4, and then every 4 weeks.

In some embodiments of the disclosed methods, uses, pharmaceutical compositions. and kits, the patient is administered the IL-17 antibody or antigen-binding fragment thereof during week 0, 1, 2, 3, 4, 8 and 12.

In some embodiments of the disclosed methods, uses, pharmaceutical compositions, and kits, the patient is administered the IL-17 antibody or antigen-binding fragment thereof weekly during week 0, 1, 2, 3, and 4, and then every 4 weeks thereafter, for a total treatment duration of at least three months.

In some embodiments of the disclosed methods, uses, pharmaceutical compositions. and kits, prior to treatment with the IL-17 antibody or antigen-binding fragment thereof, the patient failed to respond to, had an inadequate response to, or was intolerant to a prior tendinopathy treatment selected from the group consisting of local steroid injection into the affected tendon, treatment with an NSAID, treatment with acetaminophen. physiotherapy, and combinations thereof.

In some embodiments of the disclosed methods, uses, pharmaceutical compositions, and kits, the patient is undergoing physiotherapy treatment, the patient is ineligible for tendinopathy surgery, and prior to treatment with the IL-17 antibody or antigen-binding fragment thereof, the patient failed to respond to, had an inadequate response to, or was intolerant to a prior tendinopathy treatment selected from the group consisting of local steroid injection into the affected tendon, treatment with an NSAID, treatment with acetaminophen, and combinations thereof.

In some embodiments of the disclosed methods, uses, pharmaceutical compositions, and kits, the patient is undergoing physiotherapy treatment, the patient is eligible for tendinopathy surgery, and prior to treatment with the IL-17 antibody or antigen-binding fragment thereof, the patient failed to respond to, had an inadequate response to, or was intolerant to a prior tendinopathy treatment selected from the group consisting of local steroid injection into the affected tendon, treatment with an NSAID, treatment with acetaminophen, and combinations thereof.

In some embodiments of the disclosed methods, uses, pharmaceutical compositions, and kits, the patient has overuse tendinopathy.

In some embodiments of the disclosed methods, uses, pharmaceutical compositions, and kits, the patient has sub-acute tendinopathy.

In some embodiments of the disclosed methods, uses, pharmaceutical compositions, and kits, treatment with the IL-17 antibody or antigen-binding fragment thereof reduces progression to chronic tendinopathy.

In some embodiments of the disclosed methods, uses, pharmaceutical compositions, and kits, the patient has chronic tendinopathy.

In some embodiments of the disclosed methods, uses, pharmaceutical compositions, and kits, the patient has active tendinopathy.

In some embodiments of the disclosed methods, uses, pharmaceutical compositions, and kits, the patient has a partially-tom tendon.

In some embodiments of the disclosed methods, uses, pharmaceutical compositions. and kits, treatment with the IL-17 antibody or antigen-binding fragment thereof reduces progression to a fully-tom tendon.

In some embodiments of the disclosed methods, uses, pharmaceutical compositions. and kits, the patient has plantar fasciitis. Achilles tendinopathy, patellar tendinopathy, rotator cuff tendinopathy, jumper's knee, lateral epicondylitis, medial epicondylitis, supraspinatus syndrome, or any combination thereof.

In some embodiments of the disclosed methods, uses, pharmaceutical compositions, and kits, the patient experiences at least a 200% decrease in pain, at least a 20% decrease in inflammation, at least 20% improved tendon regeneration and/or repair, and/or at least 20% improved movement of the affected tendon following treatment with the IL-17 antibody or antigen-binding fragment thereof.

In some embodiments of the disclosed methods, uses, pharmaceutical compositions, and kits, following treatment with the IL-17 antibody or antigen-binding fragment thereof, the patient experiences at least a 20% decrease in pain as determined by a VAS score.

In some embodiments of the disclosed methods, uses, pharmaceutical compositions, and kits, the patient has rotator cuff tendinopathy, and following treatment with the IL-17 antibody or antigen-binding fragment thereof, the patient experiences at least a 20% improvement in shoulder-related quality of life (QoL), as determined by a WORC score, a QuickDASH score, or an ASES score.

In some embodiments of the disclosed methods, uses, pharmaceutical compositions, and kits, following treatment with the IL-17 antibody or antigen-binding fragment thereof, the patient experiences at least 20% improvement overall, as determined by a PGA score.

In some embodiments, the disclosed methods, uses, pharmaceutical compositions, and kits, further include administering a steroid, an NSAID, or acetaminophen to the patient.

In some embodiments of the disclosed methods, uses, pharmaceutical compositions, and kits, following treatment with the IL-17 antibody or antigen-binding fragment thereof, the patient has a reduced need for physiotherapy or the patient has reduced tendinopathy symptoms, thereby improving the efficacy of physiotherapy.

In some embodiments of the disclosed uses, methods and kits, the IL-17 antagonist is an IL-17 antibody or antigen-binding fragment thereof. In some embodiments of the disclosed uses, methods and kits, the IL-17 antibody or antigen-binding fragment thereof is selected from the group consisting of; a) an IL-17 antibody or antigen-binding fragment thereof that binds to an epitope of IL-17 comprising Leu74, Tyr85, His86, Met87, Asn88, Val 124, Thr125, Prol26, Ile127, Val 128, His129; b) an IL-17 antibody or antigen-binding fragment thereof that binds to an epitope of IL-17 comprising Tyr43, Tyr44, Arg46, Ala79, Asp80; c) an IL-17 antibody or antigen-binding fragment thereof that binds to an epitope of an IL-17 homodimer having two mature IL-17 protein chains, said epitope comprising Leu74, Tyr85, His86, Met87, Asn88, Val124, Thr125, Prol26, Ile127, Val128, His129 on one chain and Tyr43, Tyr44, Arg46, A1a79, Asp80 on the other chain; d) an IL-17 antibody or antigen-binding fragment thereof that binds to an epitope of an IL-17 homodimer having two mature IL-17 protein chains, said epitope comprising Leu74, Tyr85, His86, Met87, Asn88, Val124, Thr125, Prol26, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM (e.g., about 200 pM), and wherein the IL-17 antibody or antigen-binding fragment thereof has an in vivo half-life of about 23 to about 35 days (e.g., about 27 days): and e) an IL-17 antibody or antigen-binding fragment thereof comprising: i) an immunoglobulin heavy chain variable domain (Vii) comprising the amino acid sequence set forth as SEQ ID NO:8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10: iv) an immunoglobulin V_{H} domain comprising the hypervariable regions set forth as SEQ ID NO:1. SEQ ID NO:2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO:11, SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin Vii domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO:14: x) an immunoglobulin heavy chain comprising the amino acid sequence set forth as SEQ ID NO:15: or xi) an immunoglobulin light chain comprising the amino acid sequence set forth as SEQ ID NO:14 and an immunoglobulin heavy chain comprising the amino acid sequence set forth as SEQ ID NO:15.

In some embodiments of the disclosed uses, methods and kits, the IL-17 antibody or antigen-binding fragment thereof is secukinumab (AIN457), a high-affinity recombinant, fully human monoclonal anti-human interleukin-17A antibody of the IgG₁/κ-class.

Also disclosed herein are methods, uses, pharmaceutical compositions, and kits, for treating a patient having active overuse tendinopathy, comprising administering to the patient about 300 mg of secukinumab by subcutaneous injection at weeks 0, 1, 2, 3, and 4, and then every four weeks thereafter, for a total treatment duration of at least three months.

Also disclosed herein are methods, uses, pharmaceutical compositions, and kits, for treating a patient having active overuse tendinopathy, comprising administering to the patient about 150 mg of secukinumab by subcutaneous injection at weeks 0, 1, 2, 3, and 4, and then every four weeks thereafter, for a total treatment duration of at least three months.

In some embodiments of the disclosed methods, uses, pharmaceutical compositions, and kits, prior to treatment with secukinumab, the patient failed to respond to, had an inadequate response to, or was intolerant to a prior tendinopathy treatment selected from the group consisting of local steroid injection into the affected tendon, treatment with an NSAID, treatment with acetaminophen, physiotherapy, and combinations thereof.

### BRIEF DESCRIPTION OF THE FIGURE

FIG. 1 shows rat tail tendon fascile biomechanics. Rat tail tendon fascicles were cultured for 8 days in 1.67 nM IL-17A (N=1; 4 fascicles per group).

FIG. 2 shows levels of IL-6 and CXCL1 mRNA in rat Achilles tendon cells after 72 hours treatment with 1L-17A.

FIG. 3 shows that the antagonistic IL-17A antibody, BZN035, inhibits IL-17A induced IL-6 and CXCL1 mRNA in rat Achilles tenocytes.

FIG. 4 provides the tendinopathy clinical study design for Example 3. The study consists of a 4-week screening period, a 2-week run-in period, a 12-week treatment period and a 12-week follow-up period after last treatment. Note, due to a protocol change, the pre-treatment periods were modified as follows: screening Day −49 to Day −22, run-in Day −21 to Day −8, baseline Day −7 to Day −1.

### DETAILED DESCRIPTION OF THE DISCLOSURE

As used herein, “tendinopathy” is a term used to describe a complex multi-faceted pathology of the tendon characterized by pain, decline in function, and reduced exercise tolerance (Millar et al. (2017), supra). It is clinically diagnosed after gradual onset of activity-related pain, decreased function, and sometimes localized swelling, and clinical examination reveals pain with stretching and palpation of the pathological area. Ultrasonography and MRI are helpful in diagnosing active tendinopathy. Tendinopathy can occur in almost any tendon (e.g., achilles, patella, infraspinatus, epicondyle, adductor, plantar fasciopathy, subscapularis, teres minor, supraspinatus, wrist extensors, wrist flexor, hip, gluteal, etc.). As used herein, the term “tendinopathy” includes all locations and forms of tendinopathy, e.g., plantar fasciitis, Achilles tendinopathy, patellar tendinopathy, rotator cuff (infraspinatus, teres minor, supraspinatus and subscapularis) tendinopathy, tennis elbow (lateral epicondylitis), golfer's elbow (medial epicondylitis), hamstring tendinopathy, jumper's knee, supraspinatus syndrome, etc. It includes single location tendinopathy, as well as multiple-location tendinopathy.

Tendinopathy patients may have mid-substance (midportion of tendon) or enthesial (insertional) tendinopathy. As used herein, the term “tendinopathy” excludes tendon conditions arising from systemic inflammatory disease, and includes those tendon conditions developing due to injury or overuse. Tendinopathy patients may have acute, subacute, or chronic disease (Blazina et al. (1973) Orthop. Clin. North Am. 4, 665-678). Historically the terms ‘tendinitis’ and ‘tendinosis’ have interchanged with the term ‘tendinopathy’, however, these definitions are now included in the spectrum of human tendon disorders (‘tendinopathy’).

As used herein “overuse tendinopathy” refers to tendinopathy characterized by pain and tenderness at rest and/or at movement with decreased range of motion. The tendon might be swollen. Overuse tendinopathy commonly results from continuous and repeated use of the tendon, and often occurs in workers and recreational athletes. “Overuse tendinopathy” thus differs from the enthesitis found in system inflammatory diseases, e.g., psoriatic arthritis or ankylosing spondylitis. In some embodiments, the patient has overuse tendinopathy.

As used herein “the affected tendon” refers to the tendon in which a patient has tendinopathy.

As used herein, “chronic tendinopathy” and the like refers to tendinopathy that has been present for at least 6 weeks, preferably at least 12 weeks. In a clinical setting, tendinopathy is considered chronic when physiotherapy. NSAIDs and steroids have failed and pain and decreased range of motion persists. In some embodiments, the patient has overuse tendinopathy.

As used herein, the phrase “active tendinopathy” means that the patient currently experiences tendinopathy. In some embodiments, the patient has active tendinopathy, e.g., active chronic overuse tendinopathy.

As used herein, the phrase “partially-tom tendon” refers to a tendon injury (rupture or tear) that damages the tendon, but does not completely sever it from its associated bone. In some embodiments, the patient has a partially-torn tendon, preferably no more than a 50% tear as established by, e.g., ultrasound and/or MRI (e.g., assessed using Sein MRI tendinopathy scoring system and/or Bauer tendon thickness score). In some embodiments, the patient has a partially-tom tendon.

As used herein, the phrase “fully torn tendon” refers to a tendon injury (rupture or tear) that separates all of a tendon from its associated bone.

As used herein, the term “NSAID” and the phrase “Nonsteroidal anti-inflammatory drug” refers to a drug class that groups together drugs that reduce pain, decrease fever, and, in higher doses, decrease inflammation. The most prominent members of this group of drugs are aspirin, ibuprofen and naproxen. NSAIDs include salicylates (e.g., aspirin), propionic acid derivatives (e.g., ibuprophen), acetic acid derivatives (e.g., indomethacin), enolic acid derivatives (e.g., piroxicam), anthranilic acid derivatives (e.g., mefenamic), selective COX-2 inhibitors (e.g., celecoxib), sulfonanilides (e.g., nimesulide), clonixin, licofelone and h-harpagide.

As used herein, the phrase “failed to respond to” is used to mean that a patient's symptoms were not abrogated, treated, reduced, etc. in response to a particular tendinopathy treatment. In some embodiments, the tendinopathy patient failed to respond to a prior tendinopathy treatment, e.g., an NSAID, steroid (e.g., local steroid injection into the affected tendon), acetaminophen, physiotherapy, or combinations thereof.

As used herein, the phrase “had an inadequate response to” is used to mean that a patient's symptoms were not sufficiently abrogated, treated, reduced, etc. in response to a particular tendinopathy treatment. In some embodiments, the tendinopathy patient had an inadequate response to a prior tendinopathy treatment. e.g., an NSAID, steroid (e.g., local steroid injection into the affected tendon), acetaminophen, physiotherapy, or combinations thereof.

As used herein, the phrase “intolerant to” is used to mean that a patient experienced an adverse response to a particular tendinopathy treatment. In some embodiments, the tendinopathy patient was intolerant to a prior tendinopathy treatment, e.g., an NSAID, steroid (e.g., local steroid injection into the affected tendon), acetaminophen, physiotherapy, or combinations thereof.

As used herein, “fixed dose” refers to a flat dose, i.e., a dose that is not modified based on a patient's characteristics. Thus, a fixed dose differs from, e.g., a body-surface area-based dose or a weight-based dose (typically given as mg/kg). In preferred embodiments, the doses employed in the disclosed methods, uses, indications, kits, etc. are fixed doses. In most preferred embodiments, the patient is administered fixed doses of the IL-17 antibody, e.g., a fixed dose of secukinumab, e.g., a fixed dose of about 75 mg, about 150 mg, or about 300 mg of secukinumab.

As used herein, IL-17 refers to interleukin-17A (IL-17A).

As used herein, IL-17AF refers to the heterodimer consisting of a monomer of IL-17A and IL-17F.

The term “comprising” encompasses “including” as well as “consisting,” e.g., a composition “comprising” X may consist exclusively of X or may include something additional, e.g., X+Y.

As used herein, the phrase “TNF-alpha antagonist” refers to small molecules and biological molecules capable of inhibiting, reducing and/or blocking TNF-alpha signal, transduction, and/or activity. Examples of TNF-alpha antagonists include Enbrelt (etanercept), Humira@k (adalimumab), Remicade@ (infliximab) and Simponik (golimumab).

Unless otherwise specifically stated or clear from context, as used herein, the term “about” in relation to a numerical value is understood as being within the normal tolerance in the art, e.g., within two standard deviations of the mean. Thus, “about” can be within +/−10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes naturally-occurring and whole antibodies. A naturally-occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Exemplary antibodies include secukinumab (Table 1) and ixekizumab (U.S. Pat. No. 7,838,638).

The term “antigen-binding fragment” of an antibody, as used herein, refers to fragments of an antibody that retain the ability to specifically bind to an antigen (e.g., IL-17). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, Vii. CL and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody: a dAb fragment (Ward et al., 1989 Nature 341:544-546), which consists of a V_{H} domain, and an isolated CDR. Exemplary antigen-binding sites include the CDRs of secukinumab as set forth in SEQ ID NOs: 1-6 and 11-13 (Table 1), preferably the heavy chain CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using techniques known to those of skill in the art.

An “isolated antibody”, as used herein, refers to an antibody that is substantially free of other antibodies having different antigenic specificities (e.g., an isolated antibody that specifically binds IL-17 is substantially free of antibodies that specifically bind antigens other than IL-17). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed processes and compositions, the IL-17 antibody is a human antibody, an isolated antibody, and/or a monoclonal antibody.

The term “IL-17” refers to IL-17A, formerly known as CTLA8, and includes wild-type IL-17A from various species (e.g., human, mouse, and monkey), polymorphic variants of IL-17A, and functional equivalents of IL-17A. Functional equivalents of IL-17A according to the present disclosure preferably have at least about 65%, 75%, 85%, 95%, 96%, 97%, 98%, or even 99% overall sequence identity with a wild-type IL-17A (e.g., human IL-17A), and substantially retain the ability to induce IL-6 production by human dermal fibroblasts.

The term “K_{D}” is intended to refer to the dissociation rate of a particular antibody-antigen interaction. The term “K_{D}”, as used herein, is intended to refer to the dissociation constant, which is obtained from the ratio of K_{d} to K₁ (i.e., K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system. In some embodiments, the IL-17 antibody or antigen-binding fragment thereof, e.g., secukinumab, binds human IL-17 with a K_{D} of about 1-250 pM, preferably about 100-200 pM (e.g., about 200 pM).

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IL-17 of various species are known in the art, including for example, ELISAs, western blots and RIAs. The binding kinetics (e.g., binding affinity) of the antibodies also can be assessed by assays known in the art, such as by Biacore® analysis.

An antibody that “inhibits” one or more of these IL-17 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IL-17 activity affects a statistically significant decrease, e.g., by at least about 10% of the measured parameter, by at least 50%, 80% or 90%, and in certain embodiments of the disclosed methods and compositions, the IL-17 antibody used may inhibit greater than 95%, 98% or 99% of IL-17 functional activity.

“inhibit IL-6” as used herein refers to the ability of an IL-17 antibody or antigen-binding fragment thereof (e.g., secukinumab) to decrease IL-6 production from primary human dermal fibroblasts. The production of IL-6 in primary human (dermal) fibroblasts is dependent on IL-17 (Hwang et al., (2004) Arthritis Res Ther; 6:R120-128). In short, human dermal fibroblasts are stimulated with recombinant IL-17 in the presence of various concentrations of an IL-17 binding molecule or human IL-17 receptor with Fc part. The chimeric anti-CD25 antibody Simulect®(basiliximab) may be conveniently used as a negative control. Supematant is taken after 16 h stimulation and assayed for IL-6 by ELISA. An IL-17 antibody or antigen-binding fragment thereof, e.g., secukinumab, typically has an IC₅₀ for inhibition of IL-6 production (in the presence 1 nM human IL-17) of about 50 nM or less (e.g., from about 0.01 to about 50 nM) when tested as above, i.e., said inhibitory activity being measured on IL-6 production induced by hu-IL-17 inhuman dermal fibroblasts. In some embodiments of the disclosed methods and compositions, IL-17 antibodies or antigen-binding fragments thereof. e.g., secukinumab, and functional derivatives thereof have an IC₅₀ for inhibition of IL-6 production as defined above of about 20 nM or less, more preferably of about 10 nM or less, more preferably of about 5 nM or less, more preferably of about 2 nM or less, more preferably of about 1 nM or less.

The term “derivative”, unless otherwise indicated, is used to define amino acid sequence variants, and covalent modifications (e.g., pegylation, deamidation, hydroxylation, phosphorylation, methylation, etc.) of an IL-17 antibody or antigen-binding fragment thereof, e.g., secukinumab, according to the present disclosure, e.g., of a specified sequence (e.g., a variable domain). A “functional derivative” includes a molecule having a qualitative biological activity in common with the disclosed IL-17 antibodies. A functional derivative includes fragments and peptide analogs of an IL-17 antibody as disclosed herein. Fragments comprise regions within the sequence of a polypeptide according to the present disclosure, e.g., of a specified sequence. Functional derivatives of the IL-17 antibodies disclosed herein (e.g., functional derivatives of secukinumab) preferably comprise V₁₁ and/or V_{L} domains that have at least about 65%, 75%, 85%, 95%, 96%, 97%, 98%, or even 99% overall sequence identity with the Vi and/or V_{L} sequences of the IL-17 antibodies and antigen-binding fragments thereof disclosed herein, and substantially retain the ability to bind human IL-17 or, e.g., inhibit IL-6 production of IL-17 induced human dermal fibroblasts.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IL-17 antibody (e.g., a derivative of secukinumab, e.g., a secukinumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary. to achieve the maximum percent identity, and not considering any conservative substitutions as part of the sequence identity. Neither N- or C-terminal extensions nor insertions shall be construed as reducing identity. Methods and computer programs for the alignment are known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453): or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure. Amino acid sequence variants of an antibody according to the present disclosure, e.g., of a specified sequence, still have the ability to bind the human IL-17 or, e.g., inhibit IL-6 production of IL-17 induced human dermal fibroblasts. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an IL-17 binding molecule or another agent, is used to refer to delivery of that compound to a patient by any route.

As used herein, a “therapeutically effective amount” refers to an amount of an IL-17 antagonist, e.g., IL-17 binding molecule (e.g., IL-17 antibody or antigen-binding fragment thereof. e.g., secukinumab) or IL-17 receptor binding molecule (e.g., IL-17 antibody or antigen-binding fragment thereof) that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., an IL-17 antagonist, e.g., secukinumab) administered alone, the term refers to that ingredient alone. When applied to a combination, the term refers to combined amounts of the active ingredients that result in the therapeutic effect, whether administered in combination, serially or simultaneously.

The term “treatment” or “treat” is herein defined as the application or administration of an IL-17 antibody according to the disclosure, for example, secukinumab or ixekizumab, or a pharmaceutical composition comprising said anti-IL-17 antibody, to a subject or to an isolated tissue or cell line from a subject, where the subject has a particular disease (e.g., tendinopathy), a symptom associated with the disease (e.g., tendinopathy), or a predisposition towards development of the disease (e.g., tendinopathy) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating, and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology.

### IL-17 Antagonists

The various disclosed processes, kits, uses and methods utilize an IL-17 antagonist. IL-17 antagonists are capable of blocking, reducing and/or inhibiting IL-17 signal, activity and/or transduction. Examples of IL-17 antagonists include e.g., IL-17 binding molecules (e.g., soluble IL-17 receptors, IL-17 antibodies or antigen-binding fragments thereof, e.g., secukinumab and ixekizumab) and IL-17 receptor binding molecules (e.g., IL-17 receptor antibodies or antigen-binding fragments thereof, e.g., broadalumab). In some embodiments, the IL-17 antagonist is an IL-17 binding molecule, preferably an IL-17 antibody or antigen-binding fragment thereof. IL-17 antibodies and antigen-binding fragment thereof as used herein can be fully-human, CDR-grafted, or chimeric. It is preferable that the constant region domains of an antibody or antigen-binding fragment thereof for use in the disclosed methods, uses, kits, etc. preferably comprise suitable human constant region domains, for instance as described in “Sequences of Proteins of Immunological Interest”, Kabat E. A. et al, US Department of Health and Human Services, Public Health Service, National Institute of Health.

Particularly preferred IL-17 antibodies or antigen-binding fragments thereof used in the disclosed methods are human antibodies, especially secukinumab as described in Examples 1 and 2 of WO 2006/013107, which is incorporated by reference herein in its entirety. Secukinumab is a recombinant high-affinity, fully human monoclonal anti-human interleukin-17A (IL-17A, IL-17) antibody of the IgG₁/kappa isotype. Secukinumab has a high affinity for IL-17, i.e., a K_{D} of about 100-200 pM (e.g., about 200 pM), an IC₅₀ for in vitro neutralization of the biological activity of about 0.67 nM human IL-17A of about 0.4 nM, and a half-life of about 4 weeks.

For ease of reference, the amino acid sequences of the hypervariable regions of the secukinumab monoclonal antibody, based on the Kabat definition and as determined by the X-ray analysis and using the approach of Chothia and coworkers, is provided in Table 1, below.

TABLE 1
Amino acid sequences of the hypervariable regions of secukinumab.
The DNA encoding the V_{L} of secukinumab is set forth in SEQ ID NO: 9.
The DNA encoding the V_{H} of secukinumab is set forth in
SEQ ID NO: 7.
Light-Chain

| | | |
|---|---|---|
| CDR1′ | Kabat | R-A-S-Q-S-V-S-S-S-Y-L-A (SEQ ID NO: 4) |
| | Chothia | R-A-S-Q-S-V-S-S-S-Y-L-A (SEQ ID NO: 4) |
| CDR2′ | Kabat | G-A-S-S-R-A-T (SEQ ID NO: 5) |
| | Chothia | G-A-S-S-R-A-T (SEQ ID NO: 5) |
| CDR3′ | Kabat | Q-Q-Y-G-S-S-P-C-T (SEQ ID NO: 6) |
| | Chothia | Q-Q-Y-G-S-S-P-C-T (SEQ ID NO: 6) |

Heavy-Chain

| | | |
|---|---|---|
| CDR1 | Kabat | N-Y-W-M-N (SEQ ID NO: 1) |
| CDR1-x | Chothia | G-F-T-F-S-N-Y-W-M-N (SEQ ID NO: 11) |
| CDR2 | Kabat | A-I-N-Q-D-G-S-E-K-Y-Y-V-G-S-V-K-G (SEQ ID NO: 2) |
| CDR2-x | Chothia | A-I-N-Q-D-G-S-E-K-Y-Y (SEQ ID NO: 12) |
| CDR3 | Kabat | D-Y-Y-D-I-L-T-D-Y-Y-I-H-Y-W-Y-F-D-L (SEQ ID NO: 3) |
| CDR3-x | Chothia | C-V-R-D-Y-Y-D-I-L-T-D-Y-Y-I-H-Y-W-Y-F-D-L-W-G |
| | | (SEQ ID NO: 13) |

In one embodiment, the IL-17 antibody or antigen-binding fragment thereof comprises at least one immunoglobulin heavy chain variable domain (V_{H}) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the IL-17 antibody or antigen-binding fragment thereof comprises at least one immunoglobulin light chain variable domain (V_{L}) comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6. In one embodiment, the IL-17 antibody or antigen-binding fragment thereof comprises at least one immunoglobulin heavy chain variable domain (Vii) comprising hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13.

In one embodiment, the IL-17 antibody or antigen-binding fragment thereof comprises at least one immunoglobulin V_{H} domain and at least one immunoglobulin V_{L} domain, wherein: a) the immunoglobulin V_{H} domain comprises (e.g., in sequence): i) hypervariable regions CDR1, CDR2 and CDR3, said CDR1 having the amino acid sequence SEQ ID NO:1, said CDR2 having the amino acid sequence SEQ ID NO.2, and said CDR3 having the amino acid sequence SEQ ID NO:3: or ii) hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13: and b) the immunoglobulin V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6 In one embodiment, the IL-17 antibody or antigen-binding fragment thereof comprises: a) an immunoglobulin heavy chain variable domain (V_{H}) comprising the amino acid sequence set forth as SEQ ID NO:8; b) an immunoglobulin light chain variable domain (V_{L}) comprising the amino acid sequence set forth as SEQ ID NO:10; c) an immunoglobulin Vii domain comprising the amino acid sequence set forth as SEQ ID NO:8 and an immunoglobulin V_{L} domain comprising the amino acid sequence set forth as SEQ ID NO:10; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO:4 SEQ ID NO:5 and SEQ ID NO:6; f) an immunoglobulin V_{H} domain comprising the hypervariable regions set forth as SEQ ID NO: 11, SEQ ID NO:12 and SEQ ID NO:13: g) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO4, SEQ ID NO:5 and SEQ ID NO:6: or h) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO.4, SEQ ID NO:5 and SEQ ID NO:6.

In some embodiments, the IL-17 antibody or antigen-binding fragment thereof (e.g., secukinumab) comprises the three CDRs of SEQ ID NO;10. In other embodiments, the IL-17 antibody or antigen-binding fragment thereof comprises the three CDRs of SEQ ID NO:8. In other embodiments, the IL-17 antibody or antigen-binding fragment thereof comprises the three CDRs of SEQ ID NO:10 and the three CDRs of SEQ ID NO:8. CDRs of SEQ ID NO:8 and SEQ ID NO:10 may be found in Table 1. The free cysteine in the light chain (CysL97) may be seen in SEQ ID NO:6.

In some embodiments, IL-17 antibody or antigen-binding fragment thereof comprises the light chain of SEQ ID NO: 14. In other embodiments, the IL-17 antibody or antigen-binding fragment thereof comprises the heavy chain of SEQ ID NO:15 (with or without the C-terminal lysine). In other embodiments, the IL-17 antibody or antigen-binding fragment thereof comprises the light chain of SEQ ID NO:14 and the heavy domain of SEQ ID NO:15. In some embodiments, the IL-17 antibody or antigen-binding fragment thereof comprises the three CDRs of SEQ ID NO:14. In other embodiments, IL-17 antibody or antigen-binding fragment thereof comprises the three CDRs of SEQ ID NO:15. In other embodiments, the IL-17 antibody or antigen-binding fragment thereof comprises the three CDRs of SEQ ID NO:14 and the three CDRs of SEQ ID NO:15. CDRs of SEQ ID NO:14 and SEQ ID NO:15 may be found in Table 1.

Hypervariable regions may be associated with any kind of framework regions, though preferably are of human origin. Suitable framework regions are described in Kabat EA et al, ibid. The preferred heavy chain framework is a human heavy chain framework, for instance that of the secukinumab antibody. It consists in sequence, e.g. of FR1 (amino acid 1 to 30 of SEQ ID NO:8), FR2 (amino acid 36 to 49 of SEQ ID NO:8), FR3 (amino acid 67 to 98 of SEQ ID NO:8) and FR4 (amino acid 117 to 127 of SEQ ID NO:8) regions. Taking into consideration the determined hypervariable regions of secukinumab by X-ray analysis, another preferred heavy chain framework consists in sequence of FR1-x (amino acid 1 to 25 of SEQ ID NO:8), FR2-x (amino acid 36 to 49 of SEQ ID NO:8), FR3-x (amino acid 61 to 95 of SEQ ID NO:8) and FR4 (amino acid 119 to 127 of SEQ ID NO:8) regions. In a similar manner, the light chain framework consists, in sequence, of FR1′ (amino acid 1 to 23 of SEQ ID NO:10). FR2′ (amino acid 36 to 50 of SEQ ID NO:10), FR3′ (amino acid 58 to 89 of SEQ ID NO:10) and FR4′ (amino acid 99 to 109 of SEQ ID NO:10) regions.

In one embodiment, the IL-17 antibody or antigen-binding fragment thereof (e.g., secukinumab) is selected from a human IL-17 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof which comprises a variable domain comprising, in sequence, the hypervariable regions CDR1′, CDR2′, and CDR3′ and the constant part or fragment thereof of a human light chain, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen-binding fragment thereof is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising, in sequence, the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6; and c) a peptide linker which is bound either to the N-terminal extremity of the first domain and to the C-terminal extremity of the second domain or to the C-terminal extremity of the first domain and to the N-terminal extremity of the second domain.

Alternatively, an IL-17 antibody or antigen-binding fragment thereof as used in the disclosed methods may comprise a derivative of the IL-17 antibodies set forth herein by sequence (e.g., a pegylated version of secukinumab). Alternatively, the V_{H} or V_{L} domain of an IL-17 antibody or antigen-binding fragment thereof used in the disclosed methods may have V_{H} or V_{L} domains that are substantially identical to the Vii or V_{L} domains set forth in SEQ ID NO:8 and 10. A human IL-17 antibody disclosed herein may comprise a heavy chain that is substantially identical to that set forth as SEQ ID NO: 15 and/or a light chain that is substantially identical to that set forth as SEQ ID NO:14. A human IL-17 antibody disclosed herein may comprise a heavy chain that comprises SEQ ID NO:15 and a light chain that comprises SEQ ID NO:14. A human IL-17 antibody disclosed herein may comprise: a) one heavy chain, comprising a variable domain having an amino acid sequence substantially identical to that shown in SEQ ID NO:8 and the constant part of a human heavy chain; and b) one light chain, comprising a variable domain having an amino acid sequence substantially identical to that shown in SEQ ID NO: 10 and the constant part of a human light chain.

Alternatively, an IL-17 antibody or antigen-binding fragment thereof used in the disclosed methods may be an amino acid sequence variant of the reference IL-17 antibodies set forth herein, as long as it contains CysL97. The disclosure also includes IL-17 antibodies or antigen-binding fragments thereof (e.g., secukinumab) in which one or more of the amino acid residues of the V_{H} or V_{L} domain of secukinumab (but not CysL97), typically only a few (e.g., 1-10), are changed; for instance by mutation, e.g., site directed mutagenesis of the corresponding DNA sequences. In all such cases of derivative and variants, the IL-17 antibody or antigen-binding fragment thereof is capable of inhibiting the activity of about 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50%, said inhibitory activity being measured on IL-6 production induced by hu-IL-17 in human dermal fibroblasts as described in Example 1 of WO 2006/013107.

In some embodiments, the IL-17 antibodies or antigen-binding fragments thereof, e.g., secukinumab, bind to an epitope of mature human IL-17 comprising Leu74, Tyr85, His86, Met87, Asn88, Val 124, Thr125, Prol26, Ile127, Val 128, His129. In some embodiments, the IL-17 antibody. e.g., secukinumab, binds to an epitope of mature human IL-17 comprising Tyr43. Tyr44, Arg46. Ala79, Asp80. In some embodiments, the IL-17 antibody, e.g., secukinumab, binds to an epitope of an IL-17 homodimer having two mature human IL-17 chains, said epitope comprising Leu74, Tyr85, His86, Met87, Asn88, Val124, Thrl25, Prol26, Ile127, Val128, His129 on one chain and Tyr43, Tyr44, Arg46, Ala79, Asp80 on the other chain. The residue numbering scheme used to define IL-17 epitopes herein is based on residue one being the first amino acid of the mature protein (i.e., IL-17A lacking the 23 amino acid N-terminal signal peptide and beginning with Glycine). The sequence for immature IL-17A is set forth in the Swiss-Prot entry Q16552.

In some embodiments, the IL-17 antibody has a K_{D} of about 100-200 pM. In some embodiments, the IL-17 antibody has an IC₅₀ of about 0.4 nM for in vitro neutralization of the biological activity of about 0.67 nM human IL-17A. In some embodiments, the absolute bioavailability of subcutaneously (SC) administered IL-17 antibody has a range of about 60-about 80%. e.g., about 73%, about 76%. In some embodiments, the IL-17 antibody, such as secukinumab, has an elimination half-life of about 4 weeks (e.g., about 23 to about 35 days, about 23 to about 30 days, e.g., about 30 days). In some embodiments, the IL-17 antibody (such as secukinumab) has a T. of about 7-8 days.

Other preferred IL-17 antagonists for use in the disclosed methods, kits and regimens include KHK4872 (Kyowa Hakko Kirin), ABT-122 (Abbvie), BCD-085 (JCS Biopharm), vidofludimus (4SC-101), NI-1401 (RG7624; MCAF5352A-NovImmune), ANB004 (AnaptysBio Inc.), E-036041 (Ensemble Therapeutics Corp.), Q$-IL-17 (virus-like particle-based vaccine), PRS-190 (Pieris AG) bimekizumab (UCB 4940-UCB), ALX-0761, CNTO 6785 (Janssen Pharmaceuticals), LY3074828 (Eli Lilly), LY3114062 (Eli Lilly). SCH-900117, MSB0010841 (ALX-0761-Merck), ABT-122, COVA322 (Covagen), ixekizumab and brodalumab, as well as those set forth in U.S. Pat. Nos. 9,193,788; 8,057,794; 7,767,206; 8,003,099; 8,110,191; and 7,838,638 and US Published Patent Application Nos: 20120034656 and 20110027290, which are incorporated by reference herein as to their disclosure of the sequence and/or structure of an IL-17 antagonist.

In some embodiments of the disclosed uses, methods and kits, the IL-17 antibody or antigen-binding fragment thereof is selected from the group consisting of: a) an IL-17 antibody or antigen-binding fragment thereof that binds to an epitope of IL-17 between residues Arg 55 and Trp 67; b) an IL-17 antibody or antigen-binding fragment thereof that binds to an epitope of IL-17 comprising Arg 55, Glu 57, and Trp 67; c) an IL-17 antibody or antigen-binding fragment thereof that binds to an epitope of IL-17 comprising: Arg 55. Glu 57, Trp 67, Tyr 62, and Arg 101; d) an IL-17 antibody or antigen-binding fragment thereof that binds to an epitope of IL-17 comprising Arg 55. Glu 57, Trp 67, Tyr 62, Arg 101, Pro 59, Ser 64 and Val 65; e) an IL-17 antibody or antigen-binding fragment thereof that binds to an epitope of IL-17 comprising Arg 55. Glu 57, Trp 67, Tyr 62, Arg 101, Pro 59, Ser 64, Val 65, Val 22*, Leu 26, Asp 58, Glu 60, Pro 63, Pro 107, Phe 110, and Lys 114*, where amino acids marked with (*) designate a residue contributed by the second IL-17 subunit of the IL-17A homodimer, wherein the IL-17 antibody or antigen-binding fragment thereof has a K_{D} for human IL-17 of about 1-10 pM (e.g., about 6 pM), and wherein the IL-17 antibody or antigen-binding fragment thereof has an in vivo half-life of about 14-23 days, e.g., about 20 days; and f) an IL-17 antibody or antigen-binding fragment thereof comprising: i) an immunoglobulin heavy chain variable domain (V_{H}) comprising the amino acid sequence set forth as SEQ ID NO:30: ii) an immunoglobulin light chain variable domain (V_{L}) comprising the amino acid sequence set forth as SEQ ID NO:22: iii) an immunoglobulin V_{H} domain comprising the amino acid sequence set forth as SEQ ID NO:30 and an immunoglobulin V_{L} domain comprising the amino acid sequence set forth as SEQ ID NO:22; iv) an immunoglobulin V_{H} domain comprising the hypervariable regions set forth as SEQ ID NO:24, SEQ ID NO:26, and SEQ ID NO:28; v) an immunoglobulin V_{L} domain comprising the hypervariable regions set forth as SEQ ID NO:16, SEQ ID NO:18 and SEQ ID NO:20; vi) an immunoglobulin V_{H} domain comprising the hypervariable regions set forth as SEQ ID NO:25, SEQ ID NO:27 and SEQ ID NO:29: vii) an immunoglobulin Vi domain comprising the hypervariable regions set forth as SEQ ID NO:17, SEQ ID NO: 19 and SEQ ID NO:21; viii) an immunoglobulin V_{H} domain comprising the hypervariable regions set forth as SEQ ID NO:24, SEQ ID NO:26, and SEQ ID NO:28 and an immunoglobulin V_{L} domain comprising the hypervariable regions set forth as SEQ ID NO:16, SEQ ID NO:18 and SEQ ID NO:20; ix) an immunoglobulin V_{H} domain comprising the hypervariable regions set forth as SEQ ID NO:25, SEQ ID NO:27, and SEQ ID NO:29 and an immunoglobulin V_{L} domain comprising the hypervariable regions set forth as SEQ ID NO:17, SEQ ID NO:19 and SEQ ID NO:21: x) a light chain comprising SEQ ID NO:23: xi) a heavy chain comprising SEQ ID NO:31; or xii) a light chain comprising SEQ ID NO:23 and a heavy chain comprising SEQ ID NO:31.

In some embodiments of the disclosed uses, methods and kits, the IL-17 antibody or antigen-binding fragment thereof is CJM112, a high-affinity recombinant, fully human monoclonal anti-human IL-17 antibody of the IgG₁/κ-class. CJM112 also binds to IL-17AF and antagonizes this cytokine (see, e.g., U.S. Pat. No. 9,193,788, which is incorporated by reference herein in its entirety).

IL-17AF antagonists may also find use in treating tendinopathy and inducing regeneration of tendon tissue and promoting tendon repair in patients having tendinopathy. These antagonists, which include antibodies cross-reactive with IL-17A and IL-17F, and bispecific anti-IL-17A/F antibodies, FynomAbs (e.g., COVA322), nanobodies (e.g., ALX-0761), etc. may be found in US Published Patent Application Nos. 20140314763, 2013/0195872, 20160326241, U.S. Pat. Nos. 8,496,936, 8,945,553, and published PCT Application No. WO/2016/070062.

### Methods of Treatment and Uses of IL-17 Antagonists for Tendinopathy

The disclosed IL-17 antagonists. e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof), may be used in vitro, ex vivo, or incorporated into pharmaceutical compositions and administered in vivo to treat tendinopathy (e.g., human patients having tendinopathy).

It is contemplated that all responsive forms of tendinopathy may be treated using the disclosed uses, compositions, methods and kits, e.g., plantar fasciitis, Achilles tendinopathy, patellar tendinopathy, rotator cuff (infraspinatus, teres minor, supraspinatus and subscapularis) tendinopathy, tennis elbow (lateral epicondylitis), golfer's elbow (medial epicondylitis), jumper's knee, supraspinatus syndrome, etc. In preferred embodiments, the patient has rotator cuff (infraspinatus, teres minor, supraspinatus and subscapularis) tendinopathy.

In some embodiments, the patient has no tear or partial tear(s) of the affected tendon [maximum 50% tendon thickness (Bauer tendon thickness score maximum 2); AP tear size [length] maximum 10 mm (Bauer tendon length score max 2)] of the tendon (e.g., as established using ultrasound and/or MRI). MRI scoring protocols for partial tears are known (see. e.g., Bauer et al. (2014) J Orthop Surg Res. 9:128).

In some embodiments, the patient has a positive “Painful Arc Test” (see. e.g., O'Kane and Toresdahl (2014) Curr Sports Med Rep. 13(5):307-13) on examination and/or nightly pain in the affected shoulder on at least 4 out of 7 days in a given week. In some embodiments, the patient has pain in the affected shoulder (at rest or on movement) on at least 3 days out of 7 days in a given week and a score of >4 out of 10 on a pain visual analog scale (VAS).

In some embodiments, the patient's symptoms (e.g., pain, movement inhibition, and/or swelling) have been present 26 weeks. In some embodiments, the patient's symptoms (e.g., pain, movement inhibition, and/or swelling) have been present <12 months. In some embodiments, the patient's symptoms (e.g., pain, movement inhibition, and/or swelling) have been present ?6 weeks and <12 months.

In some embodiments, the patient has MRI-positive overuse (non-systemic inflammatory) rotator cuff(infraspinatus, teres minor, supraspinatus and subscapularis) tendinopathy. In some embodiments, the patient has rotator cuff (infraspinatus, teres minor, supraspinatus and subscapularis) tendinopathy without systemic inflammatory disease.

In some embodiments, the patient is refractory to (i.e., the patient failed to respond to or had an inadequate (suboptimal) response to |e.g., as determined by a VAS pain score, e.g., a VAS pain score ≥4]), or was intolerant to a prior tendinopathy treatment, e.g., steroid treatment (e.g., local steroid injection into an affected tendon), treatment with an NSAID, treatment with acetaminophen, physiotherapy, or combinations thereof.

In some embodiments, the patient previously had an inadequate response to a prior tendinopathy surgery.

Tendinopathy treatments utilizing steroids, NSAIDs, acetaminophen, physiotherapy, and combinations thereof are referred to herein as tendinopathy “standard(s) of care” treatment. A patient treated with physiotherapy is referred to as “undergoing physiotherapy treatment.”

In some embodiments of the disclosed methods, uses, pharmaceutical compositions, and kits, the patient has rotator cuff tendinopathy, is being treated using a tendinopathy standard of care treatment, and is ineligible or unwilling to undergo surgery. A patient's eligibility for surgery may be based on biochemical and/or physical characteristics, e.g., a heart health, lung health, advanced age, etc., which can be determined by a physician.

In some embodiments, the patient is steroid-naïve. In some embodiments, the patient was previously treated with local steroid injection(s) into the affected tendon. In some embodiments, the patient has multiple sites of tendinopathy.

In some embodiments, the patient is ineligible for surgery for tendinopathy.

In some embodiments, the patient is unwilling to undergo surgery for tendinopathy.

In some embodiments, the patient is ineligible for, or unwilling to undergo, surgery for tendinopathy.

In some embodiments of the disclosed methods, uses, pharmaceutical compositions, and kits, the patient is undergoing physiotherapy treatment, the patient is ineligible for tendinopathy surgery, and prior to treatment with the IL-17 antibody or antigen-binding fragment thereof, the patient failed to respond to, had an inadequate response to, or was intolerant to a prior tendinopathy treatment selected from the group consisting of local steroid injection into the affected tendon, treatment with an NSAID, treatment with acetaminophen, and combinations thereof.

In some embodiments of the disclosed methods, uses, pharmaceutical compositions, and kits, the patient is undergoing physiotherapy treatment, the patient is eligible for tendinopathy surgery, and prior to treatment with the IL-17 antibody or antigen-binding fragment thereof, the patient failed to respond to, had an inadequate response to, or was intolerant to a prior tendinopathy treatment selected from the group consisting of local steroid injection into the affected tendon, treatment with an NSAID, treatment with acetaminophen, and combinations thereof.

Response of patients to the treatments disclosed herein may be measured using patient reported outcomes (PROs), clinician reported outcomes, imaging techniques, etc. Clinician reported outcomes include, e.g., Physician's Global Assessment (PhGA) of disease activity (e.g., using a VAS score), range of movement tests (e.g., using a goniometer or digital assessment), and arm muscle strength test (e.g., using a digital manometer or rotator cuff functional index). Imaging techniques include, e.g., MRI (e.g., to determine an MRI Sein score, MRI Bauer score, supraspinatus tendon thickness, bursitis in the area of the rotator-cuff, supraspinatus tendon quality, presence of an affected bicep tendon): ultrasound; and shear wave elastography (SWE) (e.g., to assess biomechanical properties [stiffness] of the supraspinatus tendon). PROs include, e.g., the Patient's global assessment (PGA) of disease activity (e.g., using a VAS score); Western Ontario Rotator Cuff(WORC) patient reported outcome score: Disability of Arm, Shoulder and Hand Questionnaire (QuickDASH) score; American Shoulder and Elbow Surgeons Shoulder Evaluation Form (ASES) score, and EQ5D-5L score.

The WORC score is a patient reported outcome tool, uniquely developed for the rotator cuff diseases by Kirkley and co-workers (Kirkley et al. (2003) Clin J Sport Med; 13:84-92). It is a quality of life questionnaire designed for patients with rotator cuff injury. The Minimal Clinically Important Change (MCIC) in WORC has been calculated to be 275 points or 12.8% if presented in the mode of WORC %. In some embodiments, when a population of patients having rotator cuff tendinopathy is treated according to the disclosed methods (e.g., using a fixed dose of about 150 mg secukinumab or about 300 mg secukinumab given monthly, with or without a loading dose), at least 30%, at least 40%, at least 50%, at least 60%, at least 70%, at least 80% or at least 90% of patients in the population reach the MCID in WORC score. In preferred embodiments, when a population of patients having rotator cuff tendinopathy is treated according to the disclosed methods (e.g., using a fixed dose of about 150 mg secukinumab or about 300 mg secukinumab given monthly, with or without a loading dose), at least 40%, preferably at least 60%, of the patients in the population reach the MCID in WORC score.

The Quick DASH is an abbreviated form of the DASH a patient reported outcome tool, which has been developed by the American Academy of Orthopedic Surgeons along with the Institute for Work & Health (Toronto, Ontario, Canada).

The ASES score is developed by the Society of the American Shoulder and Elbow Surgeons for the assessment of shoulder function (Richards et al (1994) J Shoulder Elbow Surg 3:347-352).

EuroQuol 5D (EQ-5D-5L) (euroqol.org/eq-5d-instruments/eq-5d-51-about/) is a widely used, self-administered questionnaire designed to assess health status in adults. The purpose of the EQ-5D-5L in this study is to assess the general health status of the patients.

The MRI Sein score is used primarily to grade supraspinatus tendinosis (Sein et al. (2007) Br J Sports Med; 41(8):e9), and monitor changes in this grading over time. Tendinopathy is characterized by thickened inhomogeneous rotator cuff tendon with increased signal intensity on all pulse MRI sequences. yet not as bright as the fluid on typical T2-weighted images. Since tendonisis and partial thickness cuff injuries are prevalent in sporting activity and in middle age, another grading system, the Bauer score, can also be used for an assessment of supraspinatus partial tears and tendonosis (Bauer et al (2014) J. Orthopaedic Surg. Res 9:128). These images can also be used to measure thickness of the rotator cuff tendon and assess for presence of bursitis in the area of the rotator cuff. In some embodiments, when a population of tendinopathy are treated according to the disclosed methods, at least 40%, at least 50%, at least 60%, at least 70%, or at least 90% of the patients have less progression to tendon rupture (partial/full) as measured by MRI Bauer score. In preferred embodiments, when a population of tendinopathy are treated according to the disclosed methods, at least 50% of the patients have less progression to tendon rupture (partial/full) as measured by MRI Bauer score. In some embodiments, when a population of tendinopathy are treated according to the disclosed methods, at least 40%, at least 50%, at least 60%, at least 70%, or at least 90% of the patients have at least one grade improvement in tendon structure (i.e., decreased damage) as measured by MRI Sein score. In some embodiments, when a population of tendinopathy are treated according to the disclosed methods, at least 50% of the patients have at least one grade improvement in tendon structure (i.e., decreased damage) as measured by MRI Sein score.

Enhancement of the subacromial bursa can be assessed by the methods of Hodgson et al (2012) Br. J. Radiology; 85:1482-1487).

Improved achilles tendon pain and function can be assessed using the Victorian Institute of Sport Assessment-Achilles (VISA-A).

Improved patellar tendon pain and function can be assessed using the Victorian Institute of Sport Assessment-Patellar (VISA-P).

Improved epicondyle tendon pain and function can be assessed using the Patient-Rated Tennis Elbow Evaluation (PRTEE) Other MRI measurements may also be considered as markers of tendon “quality/integrity”, e.g., MRI techniques based on ultrashort echo-time (UTE) pulse sequences and contrast mechanisms based on T2* relaxation rate (Juras et al (2013) Eur Radiol 23:2814-2822) and/or magnetization transfer ratio (MTR) (Syha et al (2011) Fortschr Rontgenstra; 183:1043-1050).

Due to their elastic properties, tendons in the body can withstand large muscular forces with minimal deformation and loss of force transmission, by allowing energy absorption and release during motion. In tendinopathy, changes in tendon morphology and composition may result in alterations of its mechanical characteristics. Shear wave elastography (SWE) is a non-invasive ultrasonographic imaging technique, which can assess the elastic properties of soft tissue, including tendons (Chen et al (2013) J Ultrasound Med; 32: 449-455). Rotator cuff tendon stiffness can be measured by SWE.

A Visual Analogue Scale (VAS) is an instrument that measures a characteristic or attitude that is believed to range across a continuum of values. For example, the amount of pain that a patient feels ranges across a continuum from none to an extreme amount of pain. From the patient's perspective this spectrum appears continuous ± their pain does not take discrete jumps, as a categorization of none, mild, moderate and severe would suggest. The VAS pain scale is commonly used as an outcome measurement to characterize pain intensity in clinical studies. It is usually presented as a 100-mm horizontal line on which the patient's pain intensity is represented by a point between the extremes of “no pain at all” and “worst pain imaginable” (or the like). Respondents are asked to place aline perpendicular to the VAS line at the point that best indicates their pain at the present time. The tool is scored in millimeters (although scores are often recorded in tenths of centimeters using a 10-point scale), commonly a score below 40 millimeters (or 4 if recorded in centimeters) is considered desirable for chronic pain management.

In some embodiments, the patient experiences at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100/decrease in pain, decrease in inflammation, improvement in tendon regeneration and/or repair and/or improved movement following 1, 2, 3, 4, 5, 6, 7, 8, 9, 10, 11 or 12 months (preferably 1, 2 or 3 months) of treatment according to the claimed methods. In preferred embodiments, the patient experiences at least 20% decrease in pain, decrease in inflammation, improvement in tendon regeneration and/or repair, and/or improved movement following treatment according to the claimed methods.

In some embodiments, the patient experiences at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80°/o, at least 85%, at least 90%, at least 95% or 100% improvement in VAS score, WORC score, QuickDASH score, ASES score, and/or EQ5D-5L score following 1, 2, 3, 4, 5, 6, 7, 8, 9, 10, 11 or 12 months (preferably 1, 2 or 3 months) of treatment according to the claimed methods. In preferred embodiments, the patient experiences at least 20% decrease in pain, as determined by a VAS score, following treatment according to the claimed methods. In preferred embodiments, the patient experiences at least 20% improvement in shoulder-related quality of life (QoL), as determined by a WORC score, a QuickDASH score, or an ASES score, following treatment according to the claimed methods. In preferred embodiments, the patient experiences at least 20% improvement overall, as determined by a PGA score, following treatment according to the claimed methods.

The IL-17 antagonists, e.g., IL-17 binding molecules (e.g., IL-17 antibody or antigen-binding fragment thereof. e.g., secukinumab) or IL-17 receptor binding molecules (e.g., IL-17 antibody or antigen-binding fragment thereof), may be used as a pharmaceutical composition when combined with a pharmaceutically acceptable carrier. Such a composition may contain, in addition to an IL-17 antagonist, carriers, various diluents, fillers, salts, buffers, stabilizers, solubilizers, and other materials. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with the IL-17 binding molecules, or to minimize side effects caused by the IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof).

Pharmaceutical compositions for use in the disclosed methods may be manufactured in a known manner. In one embodiment, the pharmaceutical composition is provided in lyophilized form. For immediate administration it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinised blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise liquid or lyophilized formulation.

Antibodies, e.g., antibodies to IL-17, are typically formulated either in aqueous form ready for parenteral administration or as lyophilisates for reconstitution with a suitable diluent prior to administration. In some embodiments of the disclosed methods and uses, the IL-17 antagonist, e.g., IL-17 antibody, e.g., secukinumab, is formulated as a ready-to-use liquid pharmaceutical composition. Suitable lyophilisate formulations can be reconstituted in a small liquid volume (e.g., 2 ml or less) to allow subcutaneous administration and can provide solutions with low levels of antibody aggregation. The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are known. When a therapeutically effective amount of an IL-17 antagonist, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof) is administered by intravenous, cutaneous or subcutaneous injection, the IL-17 antagonist will be in the form of a pyrogen-free, parenterally acceptable solution. A pharmaceutical composition for intravenous, cutaneous, or subcutaneous injection may contain, in addition to the IL-17 antagonist, an isotonic vehicle such as sodium chloride, Ringer's solution, dextrose, dextrose and sodium chloride, lactated Ringer's solution, or other vehicle as known in the art.

In preferred embodiments of the disclosed methods, uses, kits, etc., the IL-17 binding molecule (e.g., IL-17 antibody or antigen-binding fragment thereof) or IL-17 receptor binding molecule (e.g., IL-17 antibody or antigen-binding fragment thereof) is secukinumab, which is provided in a stable liquid pharmaceutical formulation comprising about 25 mg/mL to about 150 mg/mL secukinumab, about 10 mM to about 30 mM histidine pH 5.8, about 200 mM to about 225 mM trehalose, about 0.02% polvsorbate 80, and about 2.5 mM to about 20 mM methionine, wherein the liquid formulation is not reconstituted from a lyophilisate. One preferred pharmaceutical product for use in the disclosed methods, uses, kits, etc., comprises a stable liquid formulation having 150 mg/ml secukinumab in 20 mM histidine buffer, pH 5.8, 200 mM trehalose, 0.02% polvsorbate 80 and 5 mM L-methionine, which is provided in a pre-filled syringe or an autoinjector (i.e., having 1 mL or 2 mL of the formulation).

The appropriate dosage will vary depending upon, for example, on the particular IL-17 antagonists employed, e.g., IL-17 binding molecules (e.g., IL-17 antibody or antigen-binding fragment thereof. e.g., secukinumab) or IL-17 receptor binding molecules (e.g., IL-17 antibody or antigen-binding fragment thereof), the host, the mode of administration and the nature and severity of the condition being treated, and on the nature of prior treatments that the patient has undergone. Ultimately, the attending health care provider will decide the amount of the IL-17 antagonist with which to treat each individual patient. In some embodiments, the attending health care provider may administer low doses of the IL-17 antagonist and observe the patient's response. In other embodiments, the initial dose(s) of IL-17 antagonist administered to a patient are high, and then are titrated downward until signs of relapse occur. Larger doses of the IL-17 antagonist may be administered until the optimal therapeutic effect is obtained for the patient, and the dosage is not generally increased further.

In practicing some of the methods of treatment or uses of the present disclosure, a therapeutically effective amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) is administered to a patient, e.g., a mammal (e.g., a human). While it is understood that the disclosed methods provide for treatment of tendinopathy patients using an IL-17 antagonist (e.g., secukinumab), this does not preclude that, if the patient is to be ultimately treated with an IL-17 antagonist, such IL-17 antagonist therapy is necessarily a monotherapy. Indeed, if a patient is selected for treatment with an IL-17 antagonist, then the IL-17 antagonist (e.g., secukinumab) may be administered in accordance with the methods of the disclosure either alone or in combination with other agents and therapies (e.g., other standard of care therapies for tendinopathy, e.g., steroids, NSAIDs, acetominophen, physiotherapy, etc.). Additional therapies for use in combination with the disclosed IL-17 antagonists (e.g., secukinumab) for treating tendinopathy include rest, ice, massage therapy, extracorpeal shockwave therapy (ESWT), ultrasound, laser therapy, eccentric exercise, LIPUS, electrotherapy, taping, sclerosing injections, glyceryl trinitrate, and other forms of physical therapy and physiotheraphy. Tendinopathy agents for use in combination with the disclosed IL-17 antagonists (e.g., secukinumab) for treating tendinopathy include steroids (e.g., corticosteroids, glucocorticoids, methylprednisolone, betamethasone) (oral, IV or IM), autologous blood, Platelet-rich plasma (PRP), deproteinized haemodialysate, aprotinin, polysulphated glycosaminoglycan, acetaminophen, NSAIDs (salicylates [e.g., aspirin], propionic acid derivatives [e.g., ibuprophen], acetic acid derivatives [e.g., diclofenac], enolic acid (Oxicam) derivatives [e.g., meloxicam], anthranilic acid derivatives (Fenamates) [e.g., meclofenamic acid], selective COX-2 inhibitors [e.g., celecoxib], sulfonanilides [e.g., nimesulide] and others [clonixin, licofelone, h-harpagide]). Additional tendinopathy agents for use in combination with the disclosed IL-17 antagonists (e.g., secukinumab) for treating tendinopathy include somatropin, hyaluronic acid, insulin-like growth factor I, autologous conditioned plasma, lidocaine, tetracaine patch, aethoxysclerol, polidocanol, SM04755, celestone, ketorolac, autologous mesenchymal stem cells, sodium thiosulfate, depomedrol, RCT-01, ketamine, MRX-7EAT, ketoprofen, TNF-alpha inhibitors (infliximab, adalimumab, certolizumab pegol, golimumab, etanercept), IL-1 inhibitors (anakinra, rilonacept, canakinumab), IL-23 inhibitors (ustekinumab, guselkmab), IL-17 inhibitors (ixekizumab, broadalumab), aliviador, gelol, triamcinolone acetonide, xylocaine, doxycycline, ketorolac, etoricoxib, oxycodone, bupivacaine, hydrocodone, and codeine.

When co-administered with one or more additional tendinopathy agents, an IL-17 antagonist may be administered either simultaneously with the other agent, or sequentially. If administered sequentially, the attending physician will decide on the appropriate sequence of administering the IL-17 antagonist in combination with other agents and the appropriate dosages for co-delivery. An IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) is conveniently administered parenterally, e.g., intravenously (e.g., into the antecubital or other peripheral vein), intramuscularly, or subcutaneously.

The duration of therapy using a pharmaceutical composition of the present disclosure will vary, depending on the severity of the disease being treated and the condition and personal response of each individual patient. The health care provider will decide on the appropriate duration of therapy and the timing of administration of the therapy, using the pharmaceutical composition of the present disclosure. As used herein, the phrase “total treatment duration” refers to the whole amount of time during which the patient is treated with the IL-17 antagonist, including the induction period (e.g., initial weekly dosing), if applicable. Thus, e.g., if the patient is administered the IL-17 antagonist weekly during week 0, 1, 2, 3, and 4, and then every 4 weeks for a total treatment duration of 2 months, then the patient is dosed during week 0, 1, 2, 3, 4, and 8. Similarly. e.g., if the patient is administered the IL-17 antagonist weekly during week 0, 1, 2, 3, and 4, and then every 4 weeks for a total treatment duration of 3 months, then the patient is dosed during week 0, 1, 2, 3, 4, 8, and 12.

Preferred total treatment duration is between 1-3 months, 3-6 months, 6-9 months, or 9-12 months. In some embodiments, the patient is treated for 3 months or less, e.g., 1, 2, or 3 months. In other embodiments, the patient is treated for up to 12 months, e.g., 1, 2, 3, 4, 5, 6, 7, 8, 9, 10, 11 or 12 months. Most preferred total treatment duration is 1, 2, or 3 months.

Preferred SC treatment regimens (including both induction and maintenance regimens) using secukinumab, which may be employed in treating tendinopathy patients, are provided in PCT Application No. PCT/US2011/064307 and PCT/IB2014/063902, which, as applicable, are incorporated by reference herein in their entirety.

In some embodiments, a patient may be administered a single S.C. dose of about 150 mg-about 300 mg (e.g., about 150 mg, about 300 mg) of the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof).

In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient SC at about 150 mg-about 300 mg (e.g., about 150 mg, about 300 mg) weekly during weeks 0, 1, 2, 3 and 4.

In preferred embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient SC at a fixed dose of about 150 mg-about 300 mg (e.g., about 150 mg, about 300 mg) weekly during weeks 0, 1, 2, 3 and 4, and thereafter administered to the patient SC at about 150 mg-about 300 mg (e.g., about 150 mg, about 300 mg) every 4 weeks (monthly). In this manner, the patient is dosed SC with about 150 mg-about 300 mg (e.g., about 150 mg, about 300 mg) of the IL-17 antagonist (e.g., secukinumab) during weeks 0, 1, 2, 3, 4, 8, etc.

In other embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient without a loading regimen, e.g., the antagonist may be administered to the patient SC as a fixed dose of about 150 mg-about 300 mg (e.g., about 150 mg, about 300 mg) every 4 weeks (monthly). In this manner, the patient is dosed SC with about 150 mg-about 300 mg (e.g., about 150 mg, about 300 mg) of the IL-17 antagonist (e.g., secukinumab) during weeks 0, 4, 8, 12, etc. Ideally the patient, e.g., a patient having rotator cuff tendinopathy or Achilles tendinopathy (preferably rotator cuff tendinopathy), is administered the IL-17 antagonist (e.g., secukinumab) monthly (every 4 weeks), for a total of four doses (week 0, 4, 8, and 12).

In other preferred embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient bimonthly (every 2 weeks, every other week), every other month, quarterly (every three months), biyearly (every 6 months), or yearly.

Preferred S.C. doses (e.g., fixed doses) are about 150 mg-about 3X) mg, preferably about 150 mg or about 300 mg. However, it will be understood that dose escalation may be required (e.g., during an induction and/or maintenance phase) for certain patients, e.g., patients that display inadequate response to treatment with the IL-17 antagonists, e.g., IL-17 binding molecules (e.g., IL-17 antibody or antigen-binding fragment thereof. e.g., secukinumab) or IL-17 receptor binding molecules (e.g., IL-17 receptor antibody or antigen-binding fragment thereof). Thus, SC dosages of the IL-17 antagonists, e.g., secukinumab, may be greater than about 150 mg-about 300 mg, e.g., about 175 mg, about 200 mg, about 250 mg, about 350 mg, about 400 mg, about 450 mg, about 500 mg, about 600 mg, etc. It will also be understood that dose reduction may also be required (e.g., during the induction and/or maintenance phase) for certain patients, e.g., patients that display adverse events or an adverse response to treatment with the IL-17 antagonist (e.g., IL-17 antibody or antigen-binding fragment thereof, e.g., secukinumab). Thus, dosages of the IL-17 antagonist (e.g., IL-17 antibody or antigen-binding fragment thereof, e.g., secukinumab), may be less than about 150 mg to about 300 mg SC, e.g., about 75 mg, about 100 mg, about 125 mg, about 175 mg, about 200 mg, about 250 mg, about 275 mg, etc. In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient at an initial dose (or doses) of 150 mg delivered SC, and the dose is then escalated to about 300 mg if needed, as determined by a physician. In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient at an initial dose (or doses) of 300 mg delivered SC, and the dose is then escalated to about 450 mg if needed, as determined by a physician.

The timing of dosing is generally measured from the day of the first dose of drug (which is also known as “baseline”). However, health care providers often use different naming conventions to identify dosing schedules, as shown in Table 2.

TABLE 2
Common naming conventions for dosing regimens. Bolded
items refer to the naming convention used herein.

| Week | 0/1 | 1/2 | 2/3 | 3/4 | 4/5 | 5/6 | 6/7 | 7/8 | 8/9 | 9/10 | 10/11 | etc |
|---|---|---|---|---|---|---|---|---|---|---|---|---|
| 1^{st} day | 0/1 | 7/8 | 14/15 | 21/22 | 28/29 | 35/36 | 42/43 | 49/50 | 56/57 | 63/64 | 70/71 | etc. |
| of week | | | | | | | | | | | | |

Notably, week zero may be referred to as week one by some health care providers, while day zero may be referred to as day one by some health care providers. Thus, it is possible that different physicians will designate, e.g., a dose as being given during week 3/on˜day 21, during week 3/on˜day 22, during week 4/on˜day 21, during week 4/on˜day 22, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the IL-17 antibody, regardless of whether the physician refers to a particular week as “week 1” or “week 2”. In one dosing regimen, the antibody is administered monthly during week 0, 4, 8, 12, etc. In one dosing regimen, the antibody is administered during week 0, 1, 2, 3, 4, 8, 12, etc. Some providers may refer to this regimen as weekly for five weeks and then monthly (or every 4 weeks) thereafter, beginning during week 8, while others may refer to this regimen as weekly for four weeks and then monthly (or every 4 weeks) thereafter, beginning during week 4. It will be appreciated by a skilled artisan that administering a patient an injection at weeks 0, 1, 2 and 3, followed by once monthly (every 4 weeks) dosing starting at week 4 is the same as: 1) administering the patient an injection at weeks 0, 1, 2, 3, and 4, followed by monthly dosing starting at week 8; 2) administering the patient an injection at weeks 0, 1, 2, 3 and 4 followed by dosing every 4 weeks; and 3) administering the patient an injection at weeks 0, 1, 2, 3 and 4 followed by monthly administration.

Disclosed herein are methods of treating a patient having tendinopathy, comprising administering a therapeutically effective amount of an IL-17 antibody or antigen-binding fragment thereof to a patient in need thereof, wherein the IL-17 antibody or antigen-binding fragment thereof binds to an epitope of an IL-17 homodimer having two mature IL-17 protein chains, said epitope comprising Leu74, Tyr85, His86, Met87, Asn88, Val124, Thrl25, Prol26, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

Additionally disclosed herein are therapeutically effective amounts of IL-17 antagonists (e.g., IL-17 antibody or antigen-binding fragment thereof, e.g., secukinumab) for use in treating a patient having tendinopathy, wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74. Tyr85, His86, Met87, Asn88, Val124, Thr125, Prol26, Ile127, Val128. His129 on one chain and Tyr43, Tyr44, Arg46, A1a79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in the manufacture of a medicament for treating a patient having tendinopathy, wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124. Thrl25, Prol26, Ile127. Val128, His129 on one chain and Tyr43, Tyr44, Arg46, AIa79. Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in the manufacture of a medicament for treating a patient having tendinopathy, wherein the medicament is formulated to comprise containers, each container having a sufficient amount of the IL-17 antagonist (e.g., IL-17 antibody or antigen-binding fragment thereof, e.g., secukinumab) to allow subcutaneous delivery of at least about 150 mg-about 300 mg (e.g., about 150 mg, about 300 mg) of the IL-17 antagonist (e.g., IL-17 antibody or antigen-binding fragment thereof, e.g., secukinumab) per unit dose, and further wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87. Asn88, Val124, Thr125, Prol26, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

As used herein, the phrase “container having a sufficient amount of the IL-17 antagonist to allow delivery of [a designated dose]” is used to mean that a given container (e.g., vial, pen, syringe) has disposed therein a volume of an IL-17 antagonist (e.g., as part of a pharmaceutical composition) that can be used to provide a desired dose. As an example, if a desired dose is 300 mg, then a clinician may use 2 ml from a container that contains an IL-17 antibody formulation with a concentration of 150 mg/ml, 1 ml from a container that contains an IL-17 antibody formulation with a concentration of 300 mg/ml, 0.5 ml from a container contains an IL-17 antibody formulation with a concentration of 600 mg/ml, etc. In each such case, these containers have a sufficient amount of the IL-17 antagonist to allow delivery of the desired 30) mg dose. In one embodiment, the container has disposed therein 1 ml of a formulation comprising 150 mg/ml secukinumab. In another embodiment, the container has disposed therein 2 ml of a formulation comprising 150 mg/ml secukinumab. Preferred formulations are liquid pharmaceutical compositions comprising about 25 mg/mL to about 150 mg/mL secukinumab, about 10 mM to about 30 mM histidine pH 5.8, about 200 mM to about 225 mM trehalose, about 0.02% polysorbate 80, and about 2.5 mM to about 20 mM methionine.

Additionally disclosed herein are IL-17 antagonists (e.g., IL-17 antibody or antigen-binding fragment thereof, e.g., secukinumab) for use in the manufacture of a medicament for treating a patient having tendinopathy, wherein the medicament is formulated at a dosage to allow subcutaneous delivery of about 150 mg-about 300 mg (e.g., about 150 mg, about 300 mg) of the IL-17 antagonist (e.g., IL-17 antibody or antigen-binding fragment thereof, e.g., secukinumab) to the patient, and further wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74. Tyr85, His86, Met87, Asn88, Val124. Thrl25, Prol26, lie127. Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

As used herein, the phrase “formulated at a dosage to allow [route of administration] delivery of [a designated dose]” is used to mean that a given pharmaceutical composition can be used to provide a desired dose of an IL-17 antagonist, e.g., an IL-17 antibody, e.g., secukinumab, via a designated route of administration (e.g., SC or IV). As an example, if a desired subcutaneous dose is 300 mg, then a clinician may use 2 ml of an IL-17 antibody formulation having a concentration of 150 mg/ml, 1 ml of an IL-17 antibody formulation having a concentration of 300 mg/ml, 0.5 ml of an IL-17 antibody formulation having a concentration of 600 mg/ml, etc. In each such case, these IL-17 antibody formulations are at a concentration high enough to allow subcutaneous delivery of the IL-17 antibody. Subcutaneous delivery typically requires delivery of volumes of ≤2 ml. In one embodiment, the patient is to be administered one 2 ml SC injection of a formulation containing 150 mg/ml secukinumab. In another embodiment, the patient is to be administered two 1 ml SC injections of a formulation containing 150 mg/ml secukinumab.

Disclosed herein are methods, uses, pharmaceutical compositions, and kits for inducing regeneration of tendon tissue or promoting tendon repair in a patient having tendinopathy, comprising subcutaneously administering to a patient in need thereof about 150 mg-about 300 mg of an IL-17 antibody or antigen-binding fragment thereof, wherein the IL-17 antibody or antigen-binding fragment thereof binds to an epitope of a human IL-17 homodimer having two mature human IL-17 protein chains, said epitope comprising Leu74, Tyr85, His86, Met87, Asn88, Val124, Thrl25, Prol26, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

In some embodiments of the disclosed methods, uses, pharmaceutical compositions, and kits, the patient is administered the IL-17 antibody or antigen-binding fragment thereof only once.

In some embodiments of the disclosed methods, uses, pharmaceutical compositions, and kits, the patient is administered the IL-17 antibody or antigen-binding fragment thereof weekly.

In some embodiments of the disclosed methods, uses, pharmaceutical compositions, and kits, the patient is administered the IL-17 antibody or antigen-binding fragment thereof during week 0, 1, 2, 3, and 4.

In some embodiments of the disclosed methods, uses, pharmaceutical compositions, and kits, the patient is administered the IL-17 antibody or antigen-binding fragment thereof every 4 weeks.

In some embodiments of the disclosed methods, uses, pharmaceutical compositions, and kits, the patient is administered the IL-17 antibody or antigen-binding fragment thereof for a total treatment duration of at least two months.

In some embodiments of the disclosed methods, uses, pharmaceutical compositions, and kits, the patient is administered the IL-17 antibody or antigen-binding fragment thereof for a total treatment duration of at least four months.

In some embodiments of the disclosed methods, uses, pharmaceutical compositions, and kits, the patient is administered the IL-17 antibody or antigen-binding fragment thereof weekly during week 0, 1, 2, 3, and 4, and then every 4 weeks.

In some embodiments of the disclosed methods, uses, pharmaceutical compositions, and kits, the patient is administered the IL-17 antibody or antigen-binding fragment thereof during week 0, 1, 2, 3, 4, 8 and 12.

In some embodiments of the disclosed methods, uses, pharmaceutical compositions, and kits, the patient is administered the IL-17 antibody or antigen-binding fragment thereof weekly during week 0, 1, 2, 3, and 4, and then every 4 weeks thereafter, for a total treatment duration of at least three months.

In some embodiments of the disclosed methods, uses, pharmaceutical compositions, and kits, prior to treatment with the IL-17 antibody or antigen-binding fragment thereof, the patient failed to respond to, had an inadequate response to, or was intolerant to a prior tendinopathy treatment selected from the group consisting of local steroid injection into the affected tendon, treatment with an NSAID, treatment with acetaminophen, physiotherapy, and combinations thereof.

In some embodiments of the disclosed methods, uses, pharmaceutical compositions, and kits, the patient has overuse tendinopathy.

In some embodiments of the disclosed methods, uses, pharmaceutical compositions, and kits, the patient has sub-acute tendinopathy.

In some embodiments of the disclosed methods, uses, pharmaceutical compositions, and kits, treatment with the IL-17 antibody or antigen-binding fragment thereof reduces progression to chronic tendinopathy.

In some embodiments of the disclosed methods, uses, pharmaceutical compositions, and kits, the patient has chronic tendinopathy.

In some embodiments of the disclosed methods, uses, pharmaceutical compositions, and kits, the patient has active tendinopathy.

In some embodiments of the disclosed methods, uses, pharmaceutical compositions, and kits, the patient has a partially-tom tendon.

In some embodiments of the disclosed methods, uses, pharmaceutical compositions, and kits, treatment with the IL-17 antibody or antigen-binding fragment thereof reduces progression to a fully-tom tendon.

In some embodiments of the disclosed methods, uses, pharmaceutical compositions, and kits, the patient has plantar fasciitis, Achilles tendinopathy, patellar tendinopathy, rotator cuff tendinopathy, jumper's knee, lateral epicondylitis, medial epicondylitis, supraspinatus syndrome, or any combination thereof.

In some embodiments of the disclosed methods, uses, pharmaceutical compositions, and kits, the patient experiences at least a 20% decrease in pain, at least a 20% decrease in inflammation, at least 20% improved tendon regeneration and/or repair, and/or at least 20% improved movement of the affected tendon following treatment with the IL-17 antibody or antigen-binding fragment thereof.

In some embodiments of the disclosed methods, uses, pharmaceutical compositions, and kits, following treatment with the IL-17 antibody or antigen-binding fragment thereof, the patient experiences at least a 20% decrease in pain as determined by a VAS score.

In some embodiments of the disclosed methods, uses, pharmaceutical compositions, and kits, the patient has rotator cuff tendinopathy, and following treatment with the IL-17 antibody or antigen-binding fragment thereof, the patient experiences at least a 20% improvement in shoulder-related quality of life (QoL), as determined by a WORC score, a QuickDASH score, or an ASES score.

In some embodiments of the disclosed methods, uses, pharmaceutical compositions, and kits, following treatment with the IL-17 antibody or antigen-binding fragment thereof, the patient experiences at least 20% improvement overall, as determined by a PGA score.

In some embodiments the disclosed methods, uses, pharmaceutical compositions, and kits, further include administering a steroid, an NSAID, or acetaminophen to the patient.

In some embodiments of the disclosed methods, uses, pharmaceutical compositions, and kits, following treatment with the IL-17 antibody or antigen-binding fragment thereof, the patient has a reduced need for physiotherapy or the patient has reduced tendinopathy symptoms, thereby improving the efficacy of physiotherapy.

In some embodiments of the disclosed uses, methods, pharmaceutical compositions, and kits, the IL-17 antibody or antigen-binding fragment thereof comprises: i) an immunoglobulin heavy chain variable domain (V_{H}) comprising the amino acid sequence set forth as SEQ ID NO:8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO:1, SEQ ID NO:2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO:11. SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO:14; x) an immunoglobulin heavy chain comprising the amino acid sequence set forth as SEQ ID NO:15; or xi) an immunoglobulin light chain comprising the amino acid sequence set forth as SEQ ID NO:14 and an immunoglobulin heavy chain comprising the amino acid sequence set forth as SEQ ID NO:15. In some embodiments of the disclosed uses, methods, and kits, the IL-17 antibody or antigen-binding fragment thereof is secukinumab.

Disclosed herein are also methods, uses, pharmaceutical compositions, and kits, for treating a patient having active overuse tendinopathy, comprising administering to the patient about 300 mg of secukinumab by subcutaneous injection at weeks 0, 1, 2, 3, and 4, and then every four weeks thereafter, for a total treatment duration of at least three months.

Disclosed herein are also methods, uses, pharmaceutical compositions, and kits, for treating a patient having active overuse tendinopathy, comprising administering to the patient about 150 mg of secukinumab by subcutaneous injection at weeks 0, 1, 2, 3, and 4, and then every four weeks thereafter, for a total treatment duration of at least three months. In some embodiments of the disclosed methods, uses, pharmaceutical compositions, and kits, prior to treatment with secukinumab, the patient was refractory to a prior tendinopathy treatment selected from the group consisting of local steroid injection into the affected tendon, treatment with an NSAID, treatment with acetaminophen, physiotherapy, and combinations thereof.

In some embodiments of the disclosed methods, uses, pharmaceutical compositions, and kits, the patient is undergoing physiotherapy treatment, the patient is ineligible for tendinopathy surgery, and prior to treatment with the IL-17 antibody or antigen-binding fragment thereof, the patient failed to respond to, had an inadequate response to, or was intolerant to a prior tendinopathy treatment selected from the group consisting of local steroid injection into the affected tendon, treatment with an NSAID, treatment with acetaminophen, and combinations thereof.

In some embodiments of the disclosed methods, uses, pharmaceutical compositions, and kits, the patient is undergoing physiotherapy treatment, the patient is eligible for tendinopathy surgery, and prior to treatment with the IL-17 antibody or antigen-binding fragment thereof, the patient failed to respond to, had an inadequate response to, or was intolerant to a prior tendinopathy treatment selected from the group consisting of local steroid injection into the affected tendon, treatment with an NSAID, treatment with acetaminophen, and combinations thereof.

In some embodiments of the disclosed methods, uses, pharmaceutical compositions, and kits, prior to treatment with secukinumab, the patient had a suboptimal response to a prior tendinopathy surgery.

In some embodiments of the disclosed methods, uses, pharmaceutical compositions, and kits, the patient has rotator cuff tendinopathy, is being treated using tendinopathy standard of care, and is ineligible for surgery.

In some embodiments of the disclosed methods, uses, pharmaceutical compositions, and kits, when a population of patients having rotator cuff tendinopathy is treated with secukinumab, at least 40%, preferably at least 60%, of patients reach the MCID in WORC score.

### Kits

The disclosure also encompasses kits for treating a tendinopathy patient. Such kits comprise a therapeutically effective amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) (e.g., in liquid or lyophilized form) or a pharmaceutical composition comprising a therapeutically effective amount of an IL-17 antagonist (described supra). Additionally, such kits may comprise means for administering the IL-17 antagonist (e.g., an autoinjector, a syringe and vial, a prefilled syringe, a prefilled pen) and instructions for use. These kits may contain additional therapeutic agents (described supra) for treating tendinopathy, e.g., for delivery in combination with the enclosed IL-17 antagonist, e.g., IL-17 binding molecule, e.g., IL-17 antibody, e.g., secukinumab. Such kits may also comprise instructions for administration of the IL-17 antagonist (e.g., IL-17 antibody, e.g., secukinumab) to treat the tendinopathy patient. Such instructions may provide the dose (e.g., about 150 mg-about 300 mg, e.g., about 150 mg, about 300 mg), route of administration (e.g., IV. SC. IM), regimen (e.g., weekly during week 0, 1, 2, 3, and 4; weekly during week 0, 1, 2, 3, and 4, and then every 4 weeks), and total treatment duration (e.g., 1, 2, 3, 4, 6, 8, 12 months, etc. [preferably 3-12 months, e.g., 3-6 months, e.g., 3 months]) for use with the enclosed IL-17 antagonist, e.g., IL-17 binding molecule, e.g., IL-17 antibody, e.g., secukinumab.

The phrase “means for administering” is used to indicate any available implement for systemically administering a drug to a patient, including, but not limited to, a pre-filled syringe, a vial and syringe, an injection pen, an autoinjector, an IV drip and bag, a pump, etc. With such items, a patient may self-administer the drug (i.e., administer the drug without the assistance of a physician) or a medical practitioner may administer the drug. In some embodiments, a total dose of 300 mg is to be delivered in a total volume of 2 ml having 150 mg/ml of the IL-17 antibody, e.g., secukinumab, which is disposed in a single PFS or autoinjector. In some embodiments, a total dose of 300 mg is to be delivered in a total volume of 2 ml which is disposed in two PFSs or autoinjectors, each PFS or autoinjector containing a volume of 1 ml having 150 mg/ml of the IL-17 antibody. e.g., secukinumab.

Disclosed herein are kits for use in treating a patient having tendinopathy, comprising an IL-17 antagonist (e.g., IL-17 binding molecule, e.g., IL-17 antibody or antigen-binding fragment thereof, e.g., secukinumab). In some embodiments, the kit further comprises means for administering the IL-17 antagonist to the patient. In some embodiments, the kit further comprises instructions for administration of the IL-17 antagonist, wherein the instructions indicate that the IL-17 antagonist (e.g., IL-17 binding molecule, e.g., IL-17 antibody or antigen-binding fragment thereof, e.g., secukinumab) is to be administered to the patient subcutaneously (SC) at a dose of about 150 mg-about 300 mg (e.g., about 150 mg, or about 300 mg). In some embodiments, the instructions indicate that the IL-17 antagonist (e.g., IL-17 binding molecule, e.g., IL-17 antibody or antigen-binding fragment thereof, e.g., secukinumab) is to be administered to the patient weekly during weeks 0, 1, 2, 3, and 4. In some embodiments, the instructions indicate that the IL-17 antagonist (e.g., IL-17 binding molecule, e.g., IL-17 antibody or antigen-binding fragment thereof, e.g., secukinumab) is to be administered to the patient every 4 weeks (monthly) for a total treatment duration of at least 2 months, e.g., 2, 3, 4, 5, 6, 7, 8, 9, 10, 11 or 12 months (preferably 2 or 3 months). In some embodiments, the instructions indicate that the IL-17 antagonist (e.g., IL-17 binding molecule. e.g., IL-17 antibody or antigen-binding fragment thereof, e.g., secukinumab) is to be administered to the patient weekly during weeks 0, 1, 2, 3, and 4, and every 4 weeks (monthly) thereafter, for a total treatment duration of at least 3 months, i.e., 3, 4, 5, 6, 7, 8, 9, 10, 11 or 12 months (preferably 2 or 3 months). In some embodiments, the instructions will provide for dose escalation (e.g., from a dose of about 150 mg to a higher dose of about 300 mg, or from a dose of about 300 mg to a dose of about 450 mg), as needed, to be determined by a physician.

### General

In preferred embodiments of the disclosed methods, treatments, medicaments, regimens, uses and kits, the IL-17 antagonist is an IL-17 binding molecule. In preferred embodiments, the IL-17 binding molecule is an IL-17 antibody or antigen-binding fragment thereof. In preferred embodiments of the disclosed methods, treatments, regimens, uses and kits, the IL-17 antibody or antigen-binding fragment thereof is a human antibody of the IgG₁ isotype, with a x light chain. In preferred embodiments of the disclosed methods, the antibody or antigen-binding fragment thereof is secukinumab.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference as applicable, unless otherwise indicated. The following Examples are presented in order to more fully illustrate the preferred embodiments of the disclosure. These examples should in no way be construed as limiting the scope of the disclosed subject matter, which is defined by the appended claims.

### EXAMPLES

### Example 1—Tissue Exposure and Effect of an IgG₁ Anti-IL-17 Monoclonal Antibody in Tendinopathy

In an in vivo rat disease model, tendinopathy of rotator cuff tendons was induced by unilateral surgical partial tenotomy of the supraspinatus tendon along the coronal plane. An IgG₁ anti-IL-17 monoclonal antibody (CJM112) (15 mg/kg) or vehicle was dosed subcutaneously or intravenously respectively one week or one day prior to surgical induction of tendinopathy, followed by once weekly subcutaneously or intravenously dosing for three weeks. The exposure of the antibody to rotator cuff tendon tissue and its pharmacological effects on tendinopathy inflammation and on imbalance in gait were assessed four weeks after the surgical induction of tendinopathy and one week after the last dosing of the antibody. The terminal trough exposure level of the antibody to rotator cuff tendon tissue, skeletal muscle and skin was assessed with and Enzyme-Linked Immunosorbent Assay (ELISA) following tissue homogenization and protein extraction. The effects of the antibody on tendinopathy inflammation were assessed by in-life Magnetic Resonance Imaging (MRI) (10.1136/bjsm.2006.034421) and the effects on imbalances in gait were assessed by quantification of illuminated footprints in unforced moving animals traversing a catwalk (10.1007/s 11916-014-0456-x).

Following repeated dosing, trough exposure levels of the IL-17 antibody in rotator cuff tendons were similar following subcutaneous and intravenous route of administration (500 ng/mg total protein, n=5). Trough exposure levels of the antibody to tendon tissues were similar than to skeletal muscle and lower than to skin (2′500 ng/mg total protein, n=5).

The induction of tendinopathy significantly increased the MRI T2 inflammation signal from 35 ms in normal tendons to 55 ms in tendinopathic tendons (p<0.01. Student's T-Test, n=4). The antibody reversed the increased MRI T2 tendinopathy inflammation signal (p<0.01, Student's T-Test, n=4). The induction of tendinopathy triggered imbalance in gait and significantly increased the front-hind ratio of the footprint contact area from 1 prior to induction of tendinopathy to 1.5 following induction of tendinopathy (p<0.05. ANOVA Tukey's Post Hoc Test, n=8). The antibody reversed the imbalance in gait (p<0.05, ANOVA Tukey's Post Hoc Test, n=8).

The results of this in vivo testing strongly suggest that an antagonistic IL-17 antibody will find use in treating tendinopathy.

### Example 2—Ex vivo and in vitro Example of IL17-induced Tendon Fascicle Inflammation

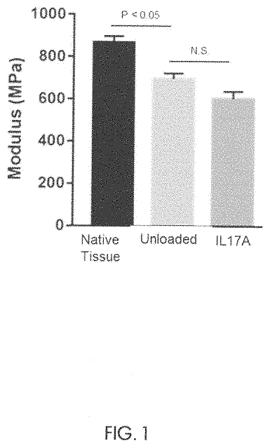

In an ex vivo rat tail tendon fascicle model, which is a model of unloading-induced tendon degeneration, RNASeq analysis revealed hallmarks of an intrinsic tendon fascicle inflammation. Unloaded fascicles showed a >10-fold upregulation of a number of cytokines, chemokines and MMPs, including IL6, CXCL1, CCL2, CCL20, CSF1-3, MMP2, 3 and 9. IL-17RA and IL-17RC were found to be highly expressed in tendon fascicles, indicating this tissue is sensitive to IL-17. Computational analysis of signaling pathways from internal databases identified five pathway hits specific for IL-17 or TH17 cell-specific signatures. Addition of recombinant IL-17A (1.67 nM) to unloaded tendon fascicles induced a further increase in the expression of cytokines, chemokines and MMPs (IL-6, CXCL-1, PTGS-2, MMP-3). Simultaneously, and in line with tendon degeneration, the tendon marker genes scleraxis and tenomodulin were dramatically downregulated. Moreover, after eight days' culture, the elastic modulus of the unloaded tendon fascicles was decreased compared to fresh tissue controls (ANOVA: p=0.0001) and IL-17A treatment induced a trend toward a further decrease (Dunnett's Post Hoc Test: p=0.0774), suggesting that inflammatory cytokines contribute to the loss of biomechanical competence (FIG. 1).

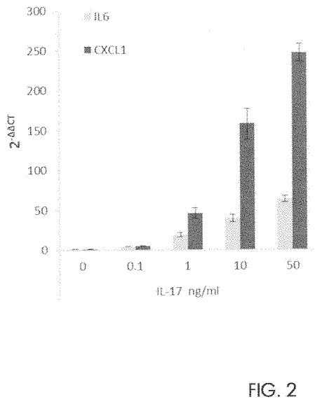

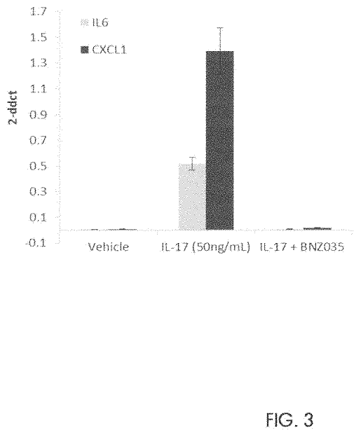

In vitro assays in rat tenocytes demonstrate that IL-17A induces dose dependent increases in IL-6 and CXCL-1 mRNA expression (FIG. 2). Furthermore, 33 nM of the rodent anti-IL-17A antibody BZN035 is able to completely block the induction of IL-17A induced IL-6 and CXCL-1 expression in rat tenocyte (FIG. 3).

### Example 3

A randomized, double-blind, placebo-controlled, parallel group, Phase II, 24-week study investigating the efficacy, safety and tolerability of AIN457 in patients with active overuse tendinopathy refractory to oral NSAIDs/acetaminophen, physiotherapy or corticosteroid injections

“Enthesitis” is the term used to describe inflammation at tendon, ligament or joint capsule insertions. It applies to diseases associated with the spondylarthritides (SpA) including AS and PsA. Enthesitis can be inflammatory or mechanically induced; the two may share common features (McGonagle D and Benjamin M (2009) Reports on Rheumatic Diseases Series 6, 4:1-6).

Neutralization of IL-17 has proven efficacy in inflammatory enthesitis in PsA and AS, as seen in studies with secukinumab. In study CAIN457F2312, enthesitis was evaluated in the subset of patients who had disease activity at baseline. In this patient population secukinumab significantly increased the percentage of patients with resolution of enthesitis compared with placebo (CAIN457F2312). Overall, the percentage of patients without resolution of enthesitis at Week 24 was 67.6%, 57.8%, 51.8%, 59.6%, and 78.5% for secukinumab 75 mg, 150 mg, 300 mg, secukinumab pooled, and placebo groups, respectively. At Week 24, these differences vs. placebo were also greater for the 150 mg and 300 mg dose groups vs placebo (p=0.0108 and p=0.0025, respectively), while 75 mg vs was similar (p=0. 1678) to placebo.

In view of the in vitro, ex vivo and in vivo tendinopathy treatment evidence shown in Examples 1 and 2, combined with the impact of IL-17 antagonism on enthesitis in PsA and AS patients, we believe that neutralization of IL-17 will reduce tendon inflammation and, perhaps more importantly, reduce tendon matrix disrepair and degeneration. Thus, secukinumab will be tested as a disease-modifying therapy for active overuse tendinopathy to induce regeneration of tenocytes and promote tendon repair.

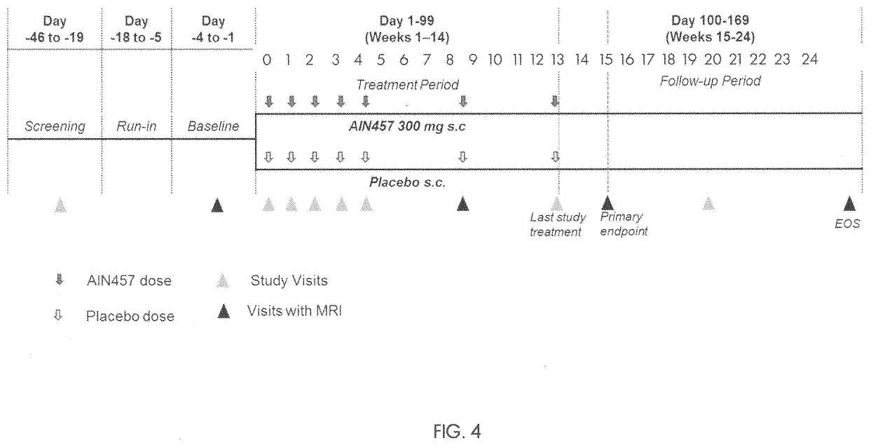

The purpose of this Phase II study is to determine the efficacy of secukinumab in treating patients with a diagnosis of overuse, non-systemic inflammatory rotator cuff (infraspinatus, teres minor, supraspinatus and subscapularis) tendinopathy and to confirm the safety and tolerability profile of secukinumab in a dose of 300 mg s.c. given at day 1 (week 0) and weekly until and including week 4 (week 1, 2, 3, and 4), thereafter additional injections at weeks 8 and 12. Efficacy of secukinumab will be evaluated at the end of week 14, based on validated patient reported outcomes (PROs) including improvement in signs and symptoms, physical function, quality of life and, range-of-movement (ROM). Changes in tendon structure reflecting tendon regeneration and repair will be investigated by MRI and shear wave elastography. Patients will be followed up without treatment from week 12 to week 24 to investigate long-term safety. Details of the study are provided below:

| Primary | To assess the efficacy of secukinumab 300 mg s.c. vs. placebo in patients | |
|---|---|---|
| Objective(s) | with overuse rotator cuff tendinopathy in relieving clinical symptoms at week | |
| | 14 | |
| Secondary | To assess the efficacy of secukinumab 300 mg s.c. vs. placebo in patients | |
| Objectives | with overuse rotator cuff tendinopathy in relieving symptoms over time. To | |
| | assess the structural changes in the rotator cuff tendinopathy over time. To | |
| | assess PK/immunogenicity in secukinumab treated patients. To confirm the | |
| | safety and tolerability of AIN457 in overuse rotator cuff tendinopathy over | |
| | time | |
| Study design | This is a randomized, double-blind, placebo-controlled, multi-center, Phase | |
| | II study of s.c. secukinumab 300 mg in approximately 100 randomized | |
| | patients with overuse rotator-cuff tendinopathy without systemic | |
| | inflammatory disease and refractory to NSAIDs/acetaminophen, | |
| | physiotherapy or corticosteroids. The patient and investigator will be blinded | |
| | throughout the study, while the sponsor will be blinded until after the analysis | |
| | of the primary endpoint. The study consists of a 4-week screening period, a | |
| | 2-week run-in period, a 12-week treatment period and a 12-week follow-up | |
| | period after last treatment (FIG. 4). | |
| | The population will consist of patients with MRI-positive unilateral overuse | |
| | (non-systemic inflammatory) shoulder tendinopathy, 18-65 years of age. | |
| | The screening period will be used to assess eligibility and to start/continue | |
| | patients on physiotherapy. In the run-in period the patient should have 2 | |
| | weeks of stable NSAID/acetaminophen intake and standardized | |
| | physiotherapy. Patients who meet the eligibility criteria at screening after the | |
| | run-in period will go through baseline evaluations. | |
| | Eligible subjects as per inclusion/exclusion criteria will be randomized to one | |
| | of the two treatment arms: seven s.c. injections of secukinumab 300 mg or | |
| | placebo in a 12-week treatment period, followed by a 12-week follow-up | |
| | period. Randomization will be stratified by the following 2 factors: | |
| | Partial tear/no tear and previous steroid injection (yes/no), in order to | |
| | achieve approximate balance between these factors in the treatment | |
| | groups. The assessments to address the primary endpoint will be performed | |
| | at 14 weeks (2 weeks after the last injection). | |
| | Patients will come to the out-patient clinic approximately 2-4 hours prior to | |
| | dosing for the evaluations. Dosing will be on-site, except for injections at 1 | |
| | and 3 weeks, that can be done either on site or by a nurse at the patient's | |
| | home. | |
| | Safety assessments will include physical examinations, ECGs, vital signs, | |
| | standard clinical laboratory evaluations (hematology, blood chemistry, and | |
| | urinalysis), adverse event and serious adverse event monitoring. | |
| Population | The population will consist of male and female patients at least 18 y of age, | |
| | but under or equal to 65 y of age at the time point of randomization with a | |
| | MRI-positive diagnosis of overuse (non-systemic inflammatory) unilateral | |
| | shoulder tendinopathy with symptoms present for at least 6 w but not more | |
| | than 12 m. | |
| Key Inclusion | Patients eligible for inclusion in this study must fulfill all of the following | |
| criteria | criteria: | |
| | Written informed consent obtained prior to all study specific screening | |
| | procedures, as close to the start of the screening period as possible | |
| | Male or non-pregnant, non-lactating female patients 18 to 65 years of | |
| | age at randomization | |
| | Presence of unilateral rotator cuff tendinopathy with: | |
| | a. | Symptoms present ≥6 weeks, but <12 months prior to |
| | | randomization |
| | b. | Tendinopathy with no more than a 50% tear as established by |
| | | ultrasound at screening and MRI at baseline: Sein MRI |
| | | tendinopathy scoring system grade I-III; with no tear or partial tear |
| | | [maximum 50% tendon thickness (Bauer tendon thickness score |
| | | maximum 2); AP length maximum 10 mm (Bauer tendon length |
| | | score max 2)]. Maximum 50% of patients with partial tear |
| | c. | Pain in the affected shoulder (at rest or on movement) on at least |
| | | 3 days out of 7 days in the past week prior to baseline and a score |
| | | of ≥4 out of 10 on a VAS pain scale |
| | d. | Positive “Painful Arc Test” on examination and/or nightly pain in |
| | | the affected shoulder on at least 4 out of 7 days in the past week |
| | | prior to baseline |

```
  The rotator-cuff tendinopathy must have been refractory to standard
  treatment defined as:
```

NSAIDs/acetaminophen
In the run-in period patients should be on a stable dose of
NSAIDs and/or acetaminophen for at least 2 weeks prior to
randomization, not exceeding - e.g.: Ibuprofen 1600 mg/d,
naproxen 1000 mg, diclofenac 105 mg/d, or diclofenac sodium
enteric-coated tablets 150 mg/d, or equivalent.
If patients cannot tolerate these doses, the maximal tolerable
dose should be used, and may be augmented with
acetaminophen/paracetamol, at doses not exceed local
guidelines or 4 g/day, whichever is lower. This medication
should also be at a stable dose for at least 2 weeks.
If patients have contraindications to NSAIDs or to
acetaminophen, these treatments can be omitted
(contraindication, drug and dose must be specified in the
eCRF).
If patients were refractory to at least 2 weeks of previous
treatment as specified in 4 i/ii, NSAIDs or acetaminophen
treatment can be omitted.

Physiotherapy

In the run in period patients should have had 2 weeks of a
standardized physiotherapy treatment before randomization

| | |
|---|---|
| Key Exclusion | History of hypersensitivity to any of the study treatments or excipients |
| criteria | or to drugs of similar chemical classes |
| | Rheumatologic, inflammatory diseases, including but not limited to: |
| | PsA, AS and RA |
| | Previous shoulder surgery in affected shoulder |
| | History of adhesive capsulitis/frozen shoulder or calcification in the |
| | tendon (in affected or contralateral shoulder) confirmed by X-Ray, |
| | historic X-Rays can be used if performed within 3 months of baseline |
| | Symptomatic osteoarthritis of the shoulder (gleno-humeral, |
| | acromioclavicular) (in affected or contralateral shoulder confirmed by |
| | X-Ray, historic X-Rays can be used if performed within 3 months of |
| | baseline |
| | Neck conditions, including but not limited to cervical spine syndrome, |
| | which in the opinion of the investigator, may explain the patient's |
| | symptoms |
| | Previous platelet rich plasma (PRP) injections within the last 12 |
| | months prior to randomization |
| Study treatment | Group 1: Secukinumab 300 mg s.c. (2 × 150 mg) |
| | Group 2: Placebo s.c. (2 injections) |
| Pharmacokinetic | PK/immunogenicity assessment |
| assessments | |
| Efficacy/PD | Patient Reported Outcomes (PROs) |
| assessments | The WORC score |
| | The QuickDASH score |
| | The ASES score |
| | The EQ5D-5L score |
| | Patient's global assessment of disease activity |
| | Clinician Reported outcome |
| | Physician's global assessment of disease activity |
| | Imaging |
| | Shoulder MRI |
| | Shear wave elastography (SWE) |
| Key safety | Key safety assessments: |
| assessments | Evaluation of AE/SAE's |
| | Physical examination |
| | Vital signs |
| | Height and weight |
| | Tuberculosis screening: QuantiFERON TB-Gold test or PPD skin test |
| | Chest X-Ray |
| | Electrocardiogram |
| | Local tolerability (Injection site reactions) |
| | Laboratory evaluations (Hematology, Clinical Chemistry, Lipid Panel, |
| | Urinalysis) |
| | Pregnancy and assessment of fertility |
| | Tolerability of secukinumab Range of Movement (ROM) |
| | Shoulder strength |
| | Sleep assessment and sleep diary |
| | DNA sampling |
| | Biomarkers assessment |
| | Immunogenicity |
| Other assessments | Range of Movement (ROM) |
| | Arm muscle strength |
| | Sleep assessment and sleep diary |
| | DNA sampling |
| | Blood biomarkers |
| Data analysis | The primary end-point (change from baseline in WORC score at Week 14) |
| | will be analyzed by repeated measures analysis of covariance including all |
| | WORC scores available. Change at Week 14 will be compared between |
| | treatments to address the primary objective. The baseline WORC score will |
| | be included as a covariate. The stratification factors partial tear/no tear and |
| | previous steroid injection (yes/no) will be included in the model as fixed |
| | effects. A 95% one-sided (90% two-sided) confidence interval for the |
| | treatment effect at Week 14 will be reported. Missing variables will be |
| | considered missing at random. |
| | Initially, models with an unstructured within-subject covariance and a |
| | separate mean for each treatment and time point will be fitted. These models |
| | may be reduced in number of covariance and mean parameters by model |
| | comparison using tools such as the BIC criterion for model fit. |

1-35. (canceled)

36: A method of inducing regeneration of tendon tissue or promoting tendon repair in a patient having tendinopathy, comprising subcutaneously administering to a patient in need thereof about 150 mg-about 300 mg of an IL-17 antibody or antigen-binding fragment thereof, wherein the IL-17 antibody or antigen-binding fragment thereof binds to an epitope of a human IL-17 homodimer having two mature human IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

37: The method according to claim 36, wherein the patient is administered the IL-17 antibody or antigen-binding fragment thereof during week 0, 1, 2, 3, and 4.

38. The method according to claim 36, wherein the patient is administered the IL-17 antibody or antigen-binding fragment thereof every four weeks.

39: The method according to claim 37, wherein the patient is administered the IL-17 antibody or antigen-binding fragment thereof weekly during week 0, 1, 2, 3, and 4, and then every four weeks.

40: The method according to claim 36, wherein, prior to treatment with the IL-17 antibody or antigen-binding fragment thereof, the patient failed to respond to, had an inadequate response to, or was intolerant to a prior tendinopathy treatment selected from the group consisting of local steroid injection into the affected tendon, treatment with an NSAID, treatment with acetaminophen, physiotherapy, and combinations thereof.

41: The method according to claim 36, wherein the patient has overuse tendinopathy, sub-acute tendinopathy, chronic tendinopathy, and/or active tendinopathy.

42: The method according to claim 36, wherein the patient has a partially-torn tendon.

43: The method according to claim 36, wherein the patient has plantar fasciitis, Achilles tendinopathy, patellar tendinopathy, rotator cuff tendinopathy, jumper's knee, lateral epicondylitis, medial epicondylitis, supraspinatus syndrome, or any combination thereof.

44: The method according to claim 43, wherein the patient has rotator cuff tendinopathy, is being treated using tendinopathy standard of care, and is ineligible for surgery.

45: The method according to claim 36, wherein the patient experiences at least a 20% decrease in pain, at least a 20% decrease in inflammation, at least 20% improved tendon regeneration and/or repair, and/or at least 20% improved movement of the affected tendon following treatment with the IL-17 antibody or antigen-binding fragment thereof.

46: The method according to claim 43, wherein the patient has rotator cuff tendinopathy, and wherein, following treatment with the IL-17 antibody or antigen-binding fragment thereof, the patient experiences at least a 20% improvement in shoulder-related quality of life (QoL), as determined by a WORC score, a QuickDASH score, or an ASES score.

47: The method according to claim 36, wherein, following treatment with the IL-17 antibody or antigen-binding fragment thereof, the patient has a reduced need for physiotherapy or the patient has reduced tendinopathy symptoms, thereby improving the efficacy of physiotherapy.

48: The method according to claim 36, wherein the IL-17 antibody or antigen-binding fragment thereof comprises:
i) an immunoglobulin heavy chain variable domain (V_{H}) comprising the amino acid sequence set forth as SEQ ID NO:8;
ii) an immunoglobulin light chain variable domain (V_{L}) comprising the amino acid sequence set forth as SEQ ID NO:10;
iii) an immunoglobulin V_{H} domain comprising the amino acid sequence set forth as SEQ ID NO:8 and an immunoglobulin V_{L} domain comprising the amino acid sequence set forth as SEQ ID NO:10;
iv) an immunoglobulin V_{H} domain comprising the hypervariable regions set forth as SEQ ID NO:1, SEQ ID NO:2, and SEQ ID NO:3;
v) an immunoglobulin V_{L} domain comprising the hypervariable regions set forth as SEQ ID NO:4, SEQ ID NO:5 and SEQ ID NO:6;
vi) an immunoglobulin V_{H} domain comprising the hypervariable regions set forth as SEQ ID NO:11, SEQ ID NO:12 and SEQ ID NO:13;
vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6;
viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6;
ix) an immunoglobulin light chain comprising the amino acid sequence set forth as SEQ ID NO:14;
x) an immunoglobulin heavy chain comprising the amino acid sequence set forth as SEQ ID NO:15; or
xi) an immunoglobulin light chain comprising the amino acid sequence set forth as SEQ ID NO:14 and an immunoglobulin heavy chain comprising the amino acid sequence set forth as SEQ ID NO:15.

49: The method according to claim 48, wherein the IL-17 antibody or antigen-binding fragment thereof is secukinumab.

50: A method of treating a patient having active overuse tendinopathy, comprising administering to the patient about 150 mg or about 300 mg of secukinumab by subcutaneous injection at weeks 0, 1, 2, 3, and 4, and then every four weeks thereafter, for a total treatment duration of at least three months.

51: The method according to claim 50, wherein, prior to treatment with secukinumab, the patient was refractory to a tendinopathy treatment selected from the group consisting of local steroid injection into the affected tendon, treatment with an NSAID, treatment with acetaminophen, physiotherapy, and combinations thereof.

52: The method according to claim 50, wherein, prior to treatment with secukinumab, the patient had a suboptimal response to a tendinopathy surgery.

53: The method according to claim 52, wherein the patient has rotator cuff tendinopathy.

54: The method according to claim 53, wherein the patient has rotator cuff tendinopathy, is being treated using tendinopathy standard of care, and is ineligible for surgery.
